(19) United States
(12) Patent Application Publication
(10) Pub. No.: US 2008/0055521 A1
(43) Pub. Date: Mar. 6, 2008

(54) METHOD OF PRODUCING OPTICAL FILM, OPTICAL FILM, POLARIZER PLATE, TRANSFER MATERIAL, LIQUID CRYSTAL DISPLAY DEVICE, AND POLARIZED ULTRAVIOLET EXPOSURE APPARATUS

(75) Inventors: Hideaki Mizutani, Kanagawa (JP); Naoyuki Kawanishi, Kanagawa (JP); Takayuki Sano, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 11/845,256

(22) Filed: Aug. 27, 2007

(30) Foreign Application Priority Data
Aug. 25, 2006 (JP) 228781/2006

(51) Int. Cl. G02F 1/1335; A61N 5/00; G02B 5/30; G02F 1/13

(52) U.S. Cl. 349/96; 250/492.1; 349/115; 349/194; 359/492

(57) ABSTRACT

A novel method of producing an optical film is disclosed. The method comprises steps (1) to (3) in this order: (1) preparing, on a surface of an alignment film, a layer of a polymerizable composition comprising a polymerizable liquid crystal compound and a dichroic polymerization initiator; (2) aligning molecules of said polymerizable liquid crystal compound in said layer in a first alignment state; and (3) irradiating said layer with polarized ultraviolet light to carry out polymerization of said polymerizable liquid crystal compound and fix molecules of said polymerizable liquid crystal compound in a second alignment state thereby to form an optically anisotropic layer,
wherein a percentage of polarized ultraviolet light having an extinction ratio ranging from 1 to 8 is not greater than 15% with respect to an energy density of polarized ultraviolet light per unit area (J/cm²).

### CROSS-REFERENCE TO RELATED APPLICATIONS

This application claims benefit of priority under 35 U.S.C. 119 to Japanese Patent Application No. 2006-228781 filed Aug. 25, 2006, and the entire content of the application is incorporated herein by reference.

### BACKGROUND OF THE INVENTION

1. Field of the Invention

The present invention relates to a method of producing an optical film, and a polarized ultraviolet exposure apparatus effectively used therefor. The present invention also relates to an optical film produced by the method, a polarizer plate using the same, a transfer material and a liquid crystal display device. In particular, the present invention relates to an optical film capable of contributing improvement in viewing angle dependence of vertically-aligned liquid crystal display devices, and a liquid crystal display device improved in the viewing angle dependence.

2. Related Art

A CRT (cathode ray tube) has been mainly employed in various display devices used for office automation (OA) equipment such as a word processor, a notebook-sized personal computer and a personal computer monitor, mobile phone terminal and television set. A liquid crystal display device (LCD) has been more and more widely used instead of a CRT, because it has a thin shape, lightweight and small electric power consumption. A liquid crystal display device comprises, at least, a liquid crystal cell and a polarizing plate. In general, a polarizing plate is produced by laminating the both surfaces of a polarizing film, which is prepared by soaking a polyvinyl alcohol film with iodine and then subjecting the same to stretching, with protective films, and, therefore, comprises a pair of protective films and a polarizing film. For example, a transmissive LCD comprises two polarizing plates disposed on both sides of a liquid crystal cell, and may further comprise one or more optical compensatory sheets. On the other hand, a reflective LCD comprises a reflecting plate, a liquid crystal cell, one or more optical compensatory sheets, and a polarizing plate which are disposed in this order. A liquid crystal cell comprises a liquid crystal layer confined between two substrates, and electrode layers for applying a voltage to the liquid crystal layer. A liquid crystal cell has ON and OFF states on the basis of the difference in alignment state of the liquid crystal layer, and can be used in any of a transmissive type, reflective type and semi-transmissive type display devices employing any ever proposed modes such as TN (Twisted Nematic), IPS (In-Plane Switching), OCB (Optically Compensatory Bend), VA (Vertically Aligned), ECB (Electrically Controlled Birefringence) and STN (Super Twisted Nematic). Color and contrast displayed by the conventional liquid crystal display device, however, vary depending on the viewing angle. Therefore, it cannot be said that the viewing angle characteristics of the liquid crystal display device is superior to those of the CRT.

In recent years, a proposal has been made on a vertically-aligned (referred to as VA-mode, hereinafter) nematic liquid crystal display device, as an LCD improved in the viewing angle dependence, using nematic liquid crystal molecules having negative dielectric anisotropy, configured as aligning the long axes thereof nearly normal to the substrates in the absence of applied voltage, and as driving them by thin-film transistors (Japanese Laid-Open Patent Publication No. H2-176625). A VA-mode LCD is characterized not only by its excellence in display performance in the front view as well as a TN-mode LCD, but also by its wide viewing angle obtained when applied with a retardation plate (optical compensation film) for compensating vieing angle. A VA-mode LCD employing a negative monoaxial retardation plate (negative c-plate) having an optical axis normal to the film plane can show a still wider viewing angle propety, and it is also known that a LCD employing a monoaxially-aligned retardation plate (positive a-plate) having an in-plane retardation of 50 nm and positive refractive index anisotropy can achieve a still more wider viewing angle (SID 97 DIGEST p. 845-848).

Increase in the number of retardation plates, however, pushes up the production cost. Bonding of a large number of films not only tends to degrade the yield ratio, but also degrades display quality due to misalignment of the angle of bonding. Use of a plurality of films increases the thickness of the display device, and thereby may raise disadvantage in terms of thinning display devices.

A positive a-plate is generally configured using a stretched film, wherein the a-plate will have a slow axis in the moving direction (MD) of the film, if the film is a longitudinally stretched film produced by a continuous moving process. In compensation of viewing angle in the VA mode, it is, however, necessary to cross the slow axis of the a-plate normal to MD, which is the direction of absorption axis of the polarizer plate, making roll-to-roll bonding impossible, and considerably raising the costs. One possible solution to this problem may be use of a so-called transversely stretched film obtained by stretching the film in the direction (TD direction) normal to MD. The transversely stretched film is, however, likely to produce distortion in the slow axis, called bowing, and therefore pushes up the cost due to poor yield ratio. Still another disadvantage is such that an adhesive layer, used for stacking the stretched films, may shrink under varied temperature and humidity, and may consequently result in failures such as separation of the films and warping. As one method of improving these problems, there has been known a method of producing an a-plate by coating rod-like liquid crystal (see Japanese Laid-Open Patent Publication No. 2000-304930).

More recently, a method of using a biaxial retardation plate, in place of a combination of c-plate and a-plate, has been proposed (SID 2003 DIGEST p. 1208-1211). Use of the biaxial retardation plate has an advantage of being capable of improving not only viewing angle dependence of contrast but also hue, but also has a disadvantage in that it is difficult for biaxial stretching adopted to producing of the biaxial retardation plate to uniformly control the axis over the entire region of the film, similarly to the transverse stretching, and so that the yield ratio cannot be raised and thereby the costs increase.

It has therefore been proposed methods of producing biaxial retardation plates without relying upon stretching, by irradiating a special cholesteric liquid crystal with polarized ultraviolet light (EP1389199 A1), or by irradiating a specific discotic liquid crystal with polarized ultraviolet light (Japanese Laid-Open Patent Publication No. 2002-6138). These methods can solve various problems ascribable to stretching.

By the way, irradiation of polarized ultraviolet light requires a polarization filter exhibiting a polarization separation performance for 380 nm or shorter wavelength ultraviolet light, and may therefore give only an energy density (UV dose) smaller than that of non-polarized light, because at least a half of the polarization components cannot be available because of an intrinsic feature of the polarizer filter. Faster speed of feeding will be necessary in order to keep high productivity, but faster speed of feeding reduces the energy density of polarized ultraviolet light, inversely proportional thereto.

Too small energy density of polarized ultraviolet light used for producing the biaxial polarizer plate and so forth may result in only a poor strength of the film after being cured, and may affect the molecular alignment for the case where alignment of the curing is associated with alignment of liquid crystal molecules, and may consequently affect optical characteristics of the resultant optical film and so forth.

As a means for aligning liquid crystal molecules by irradiating polarized light, a means for converting light partially into parallel beam or polarized beam is described in Published Japanese Translation of PCT International Publication for Patent Application No. 2001-512850 specifically. A polarized light irradiating apparatus, employing a wire-grid polarization element, improved in efficiency of use of light is described in Japanese Laid-open Patent Publication No. 2004-144884. However, for the case where polarized ultraviolet light irradiated to form the optically anisotropic layer is relevant to both of alignment of the liquid crystal molecules in the layer and curing of the layer, it is necessary to allow the liquid crystal to align and cure uniformly and thoroughly in the thickness-wise direction, for the purpose of obtaining desired levels of optical characteristics and strength of the film.

### SUMMARY OF THE INVENTION

It is therefore an object of the present invention to provide a method of producing an optical film, comprising a step of irradiating polarized ultraviolet light, capable of producing an optical film excellent in optical characteristics and strength of the film with high productivity, and to provide a polarized ultraviolet exposure apparatus suitable for the method.

It is another object of the present invention to provide a method of producing an optical film contributive to improvement in viewing angle dependence of liquid crystal display devices in particular VA-mode ones, in continuous, non-defective or less-defective, and stable manner.

It is still another object of the present invention to provide a polarizer plate having such optical film and is applicable as one component of liquid crystal display device, in particular VA-mode ones, and a transfer material making it possible to readily form an optically anisotropic layer in a liquid crystal cell.

It is still another object of the present invention to provide a liquid crystal display device, in particular VA-mode one, having a liquid crystal cell optically compensated in an exact manner, being possibly thinned, and improved in the viewing angle dependence.

In one aspect, the present invention provides a method of producing an optical film comprising steps (1) to (3) in this order:

(1) preparing, on a surface of an alignment film, a layer of a polymerizable composition comprising a polymerizable liquid crystal compound and a dichroic polymerization initiator;

(2) aligning molecules of said polymerizable liquid crystal compound in said layer in a first alignment state; and

(3) irradiating said layer with polarized ultraviolet light to carry out polymerization of said polymerizable liquid crystal compound and fix molecules of said polymerizable liquid crystal compound in a second alignment state thereby to form an optically anisotropic layer,

wherein a percentage of polarized ultraviolet light having an extinction ratio ranging from 1 to 8 is not greater than 15% with respect to an energy density of polarized ultraviolet light per unit area (J/cm²).

As embodiments of the invention, there are provided the method wherein a surface temperature of said layer in the step (3) is from (Tᵢₛₒ−50) to Tᵢₛₒ° C. (where, Tᵢₛₒ(° C.) is isotropic phase transition temperature of said polymerizable liquid crystal compound); and the method wherein, in the step (3), polarized ultraviolet light is irradiated with an energy density within a range from 200 mJ/cm² to 2 J/cm²; the method wherein the layer is irradiated with non-polarized ultraviolet light after the step (3).

In another aspect, the present invention provides a polarized ultraviolet exposure apparatus to be used in the above mentioned method, comprising an ultraviolet radiation source, a unit of converting non-polarized ultraviolet light from said radiation source into polarized ultraviolet light; and a unit of preventing an object to be irradiated from being irradiated with polarized ultraviolet light having an extinction ratio ranging from 1 to 8; an optical film produced by the above mentioned method; a polarizer plate comprising a polarizer film, and the optical film; a transfer material comprising an optical film produced according to the above mentioned method; and a photosensitive polymer layer disposed on an optically anisotropic layer of said optical film; a liquid crystal display device comprising at least one selected from the polarizer plate, the optical film and the optically anisotropic layer transferred from the transfer material; and a liquid crystal display device comprising, in a liquid crystal cell thereof, an optically anisotropic layer transferred from the transfer material.

### BRIEF DESCRIPTION OF THE DRAWINGS

FIGS. 1A and 1B are schematic drawings of an exemplary polarized ultraviolet irradiation apparatus of the present invention.

FIG. 2 is a drawing showing distribution of intensity (mW/cm²) in relation to the direction of feeding in Example 1.

FIG. 3 is a drawing showing distribution of extinction ratio in relation to the direction of feeding in Example 1.

FIG. 4 is a schematic sectional view showing an exemplary optical compensation film of the present invention.

FIGS. 5A to 5D are schematic sectional views showing an exemplary polarizer plate of the present invention.

FIGS. 6A to 6E are schematic sectional views showing an exemplary transfer material of the present invention.

FIGS. 7A to 7C are schematic sectional views showing an exemplary substrate for liquid crystal cell, produced using the transfer material of the present invention.

FIG. 8 is a schematic sectional view showing an exemplary liquid crystal display device of the present invention.

FIGS. 9A to 9C are schematic sectional views showing an exemplary liquid crystal display device containing an optically anisotropic layer transferred from a transfer material of the present invention.

FIG. 10 is a schematic drawing of the polarized ultraviolet irradiation apparatus used in Example 1.

FIG. 11 is a schematic sectional view showing a layer configuration of the liquid crystal display device produced in Example 5, together with the direction of optical axes.

FIG. 12 is a drawing showing a contrast characteristic of the liquid crystal display device produced in Example 5.

Reference numerals used in the drawings are as follows:
1 reflecting mirror
2 rod-like ultraviolet lamp
3 optical component adjusting direction of beam
4 wavelength selection filter
5 aperture intercepting light components having small extinction ratios
6 polarizer
7 irradiation surface
8 slit width
9 source lamp unit
11 support
12 optically anisotropic layer
13 alignment layer
14 photosensitive polymer layer
15 cushion layer
16 protective layer
21 polarizer layer (polarizer film)
22, 23 protective film
24 functional layer such as λ/4 plate, anti-reflecting film, etc.
25 transparent electrode layer
26 alignment layer
27 optically compensation layer
27′ patterned optically compensation layer
28 color filter layer
29 black matrix layer
30 support (also being object to be transferred)
31 liquid crystal layer
32 TFT layer
33 polarizer layer
34 protective film
35 protective film (may occasionally being optical compensation film)
36 polarizer plate
37 liquid crystal cell
41 polarizer layer
42 transparent support
43 alignment layer
44 optically anisotropic layer
45 polarizer plate protective film
46 glass substrate for liquid crystal cell
47 liquid crystal cell
48 pressure-sensitive adhesive
51 cold cathode ray tube
52 reflective sheet
53 light guide plate
54 light conditioning film such as luminance improving film, diffuser film or the like
55 liquid crystal cell
56 lower polarizer plate
57 upper polarizer plate

### DETAILED DESCRIPTION OF THE INVENTION

Paragraphs below will detail the present invention. It is to be noted that the expression “to” in this specification means a range expressed by the numerals placed therebefore and thereafter as the lower limit value and the upper limit value, respectively.

In this specification, Re(λ) and Rth(λ) represent in-plane retardation and in-thickness direction retardation at wavelength λ, respectively. Re(λ) is measured using KOBRA 21ADH or WR (from Oji Scientific Instruments), by irradiating the film with a λ-nm light in the direction of normal line of the film. Rth(λ) is calculated by KOBRA 21ADH is calculated based on the Re(λ) and plural retardation values which are measured for incoming light of a wavelength λ nm in plural directions with a variable angle with respect to the normal direction of a sample film using an in-plane slow axis, which is decided by KOBRA 21ADH, as an a tilt axis (a rotation axis); a value of hypothetical mean refractive index; and a value entered as a thickness value of the film.

In the specification, the term “substantively” with respect to angle means an angle has an allowable error within ±5′, preferably ±4′, and more preferably ±3′. The term “substantively” with respect to retardation means a retardation has an allowable error within ±5%. The term “Re is not zero” means that Re is not less than 5 nm. The measurement wavelength for refractive indexes is a visible light wavelength, unless otherwise specifically noted. It is also to be noted that the term “visible light” in the context of this specification means light of a wavelength falling within the range from 400 to 700 nm.

[Method of Producing Optical Film]

The present invention relates to a method of producing an optical film comprising the following steps (1) to (3), in this order:

(1) preparing, on a surface of an alignment film, a layer of a polymerizable composition comprising a polymerizable liquid crystal compound and a dichroic polymerization initiator;

(2) aligning molecules of the polymerizable liquid crystal compound in the layer in a first alignment state; and

(3) irradiating said layer with polarized ultraviolet light to carry out polymerization of said polymerizable liquid crystal compound and fix molecules of said polymerizable liquid crystal compound in a second alignment state thereby to form an optically anisotropic layer,

wherein the percentage of polarized ultraviolet light having an extinction ratio ranging from 1 to 8 is not greater than 15% with respect to an energy density of polarized ultraviolet light per unit area (J/cm²).

[Step (1)]

In step (1), a layer of a polymerizable composition comprising a polymerizable liquid crystal compound and a dichroic polymerization initiator is prepared on the surface of an alignment film. The layer can be prepared typically by applying a coating liquid, which is a polymerizable composition comprising a polymerizable liquid crystal compound, a dichroic polymerization initiator and optional additive(s), to a surface and drying it.

According to the invention, the polymerizable liquid crystal to be used has no limitation. In general, liquid crystal compounds can be classified into a rod-like type and a disc-shaped type on the basis of the figure thereof. Each type includes a low molecular type and a high molecular type. A high molecule generally indicates a molecule having a polymerization degree of 100 or more (Doi Masao; Polymer Physics Phase transition Dynamics, page 2 Iwanami Shoten, 1992). In the invention, although any types of liquid crystal compounds can be used, rod-like liquid crystal compounds are preferred in terms of efficient generation of in-plane retardation by polarized ultraviolet irradiation. A mixture of two types or more of the rod-like liquid crystal compounds, two types or more of the disc-shaped liquid crystal compounds, or the rod-like liquid crystal compound and disc-shaped liquid crystal compound may be used. According to the invention, at least one polymerizable liquid crystal compound is used, and at least one polymerizable liquid crystal of which molecule has two or more reactive groups is preferably used. A liquid crystal compound having no reactive group may be used in combination with the polymerizable one. In the case of the mixture, it is preferred that at least one type has two or more reactive groups in one liquid crystal molecule.

According to the invention, the discotic liquid crystal to be used has no limitation; and it may be selected from known any discotic liquid crystals. Preferable examples of the rod-like liquid crystal include azomethines, azoxy compounds, cyanobiphenyls, cyanophenyl esters, benzoate ester, cyclohexanecarboxyl phenyl esters, cyanophenylcyclohexanes, cyano-substituted phenylpyrimidines, alkoxy-substituted phenylpyrimidines, phenyldioxanes, tolans and alkenyl cyclohexyl benzonitriles. The polymerizable liquid crystal compound may be selected from low-molecular weight compounds or high-molecular weight compounds. Preferred examples of the polymerizable liquid crystal compound include compounds represented by the formula (I) shown below.
Q¹-L¹-A¹-L³-M-L⁴-A²-L²-Q² (I)

In the formula, Q¹ and Q² respectively represent a reactive group. L¹, L², L³ and L⁴ respectively represent a single bond or a divalent linking group, and it is preferred that at least one of L³ and L⁴ represents —O—CO—O—. A¹ and A² respectively represent a C₂₋₂₀ spacer group. M represents a mesogen group.

In formula (I), a reactive represented by Q¹ or Q² is a polymerizable group; and the polymerizable groups capable of addition polymerization (including ring opening polymerization) or condensation polymerization are preferred. Examples of reactive groups are shown below.

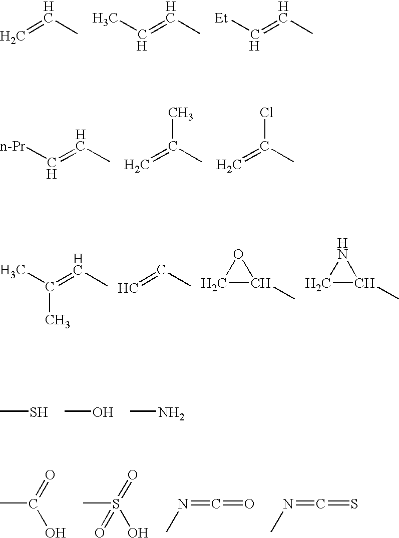

L¹, L², L³ and L⁴ independently represent a divalent linking group, and preferably represent a divalent linking group selected from the group consisting of —O—, —S—, —CO—, —NR²—, —CO—O—, —O—CO—O—, —CO—NR²—, —NR²—CO—, —O—CO—, —O—CO—NR²—, —NR²—CO—O— and —NR²—CO—NR²—. R² represents a C₁₋₇ alkyl group or a hydrogen atom. It is preferred that at least one of L³ and L⁴ represents —O—CO—O— (carbonate group). It is preferred that Q¹-L¹ and Q²-L²- are respectively CH₂═CH—CO—O—, CH₂═C(CH₃)—CO—O— or CH₂═C(Cl)—CO—O—CO—O—; and it is more preferred they are respectively CH₂═CH—CO—O—.

In the formula, A¹ and A² preferably represent a C₂₋₂₀ spacer group. It is more preferred that they respectively represent C₂₋₁₂ aliphatic group, and much more preferred that they respectively represent a C₂₋₁₂ alkylene group. The spacer group is preferably selected from chain groups and may contain at least one unadjacent oxygen or sulfur atom. And the spacer group may have at least one substituent such as a halogen atom (fluorine, chlorine or bromine atom), cyano, methyl and ethyl.

Examples of the mesogen represented by M include any known mesogen groups. The mesogen groups represented by a formula (II) are preferred.
—(—W¹-L⁵)ₙ-W² (II)

In the formula, W¹ and W² respectively represent a divalent cyclic aliphatic group, a divalent aromatic group or a divalent hetero-cyclic group; and L⁵ represents a single bond or a linking group. Examples of the linking group represented by L⁵ include those exemplified as examples of L¹ to L⁴ in the formula (I) and —CH₂—O— and —O—CH₂—. In the formula, n is 1, 2 or 3.

Examples of W¹ and W² include 1,4-cyclohexanediyl, 1,4-phenylene, pyrimidine-2,5-diyl, pyridine-2,5-diyl, 1,3,4-thiazole-2,5-diyl, 1,3,4-oxadiazole-2,5-diyl, naphtalene-2,6-diyl, naphtalene-1,5-diyl, thiophen-2,5-diyl, pyridazine-3,6-diyl. 1,4-cyclohexanediyl has two stereoisomers, cis-trans isomers, and the trans isomer is preferred. W¹ and W² may respectively have at least one substituent. Examples the substituent include a halogen atom such as a fluorine, chlorine, bromine or iodine atom; cyano; a C₁₋₁₀ alkyl group such as methyl, ethyl and propyl; a C₁₋₁₀ alkoxy group such as methoxy and ethoxy; a C₁₋₁₀ acyl group such as formyl and acetyl; a C₂₋₁₀ alkoxycarbonyl group such as methoxy carbonyl and ethoxy carbonyl; a C₂₋₁₀ acyloxy group such as acetyloxy and propionyloxy; nitro, trifluoromethyl and difluoromethyl.

Preferred examples of the basic skeleton of the mesogen group represented by the formula (II) include, but not to be limited to, these described below. And the examples may have at least one substituent selected from the above.

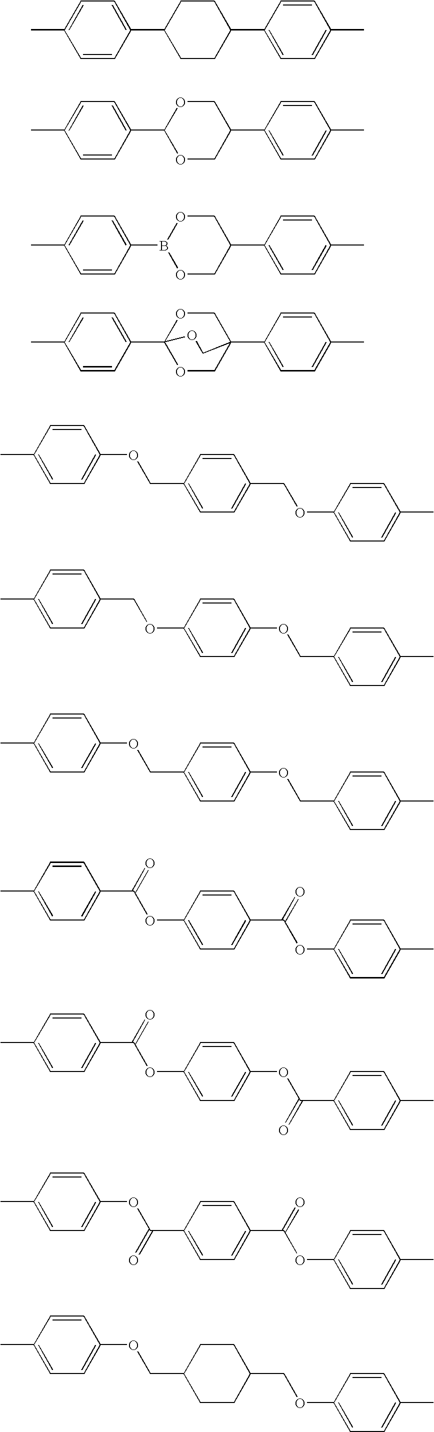

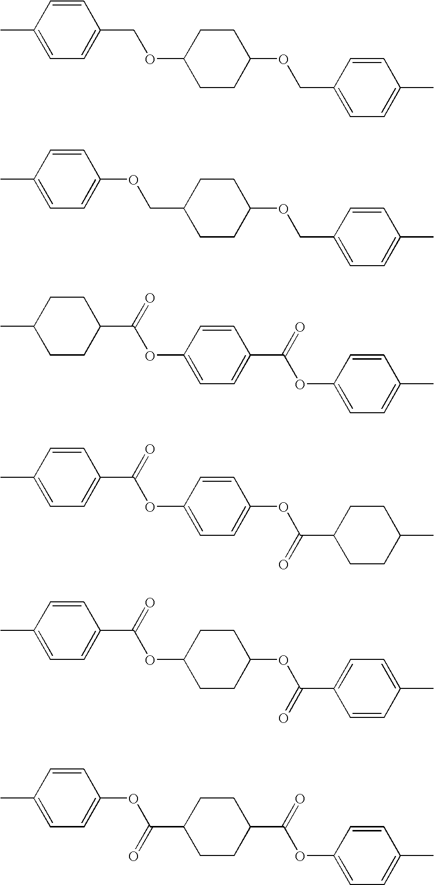

Examples the compound represented by the formula (I) include, but not to be limited to, these described below. The compounds represented by the formula (I) may be prepared according to a method described in a gazette of Tokkohyo No. hei 11-513019.

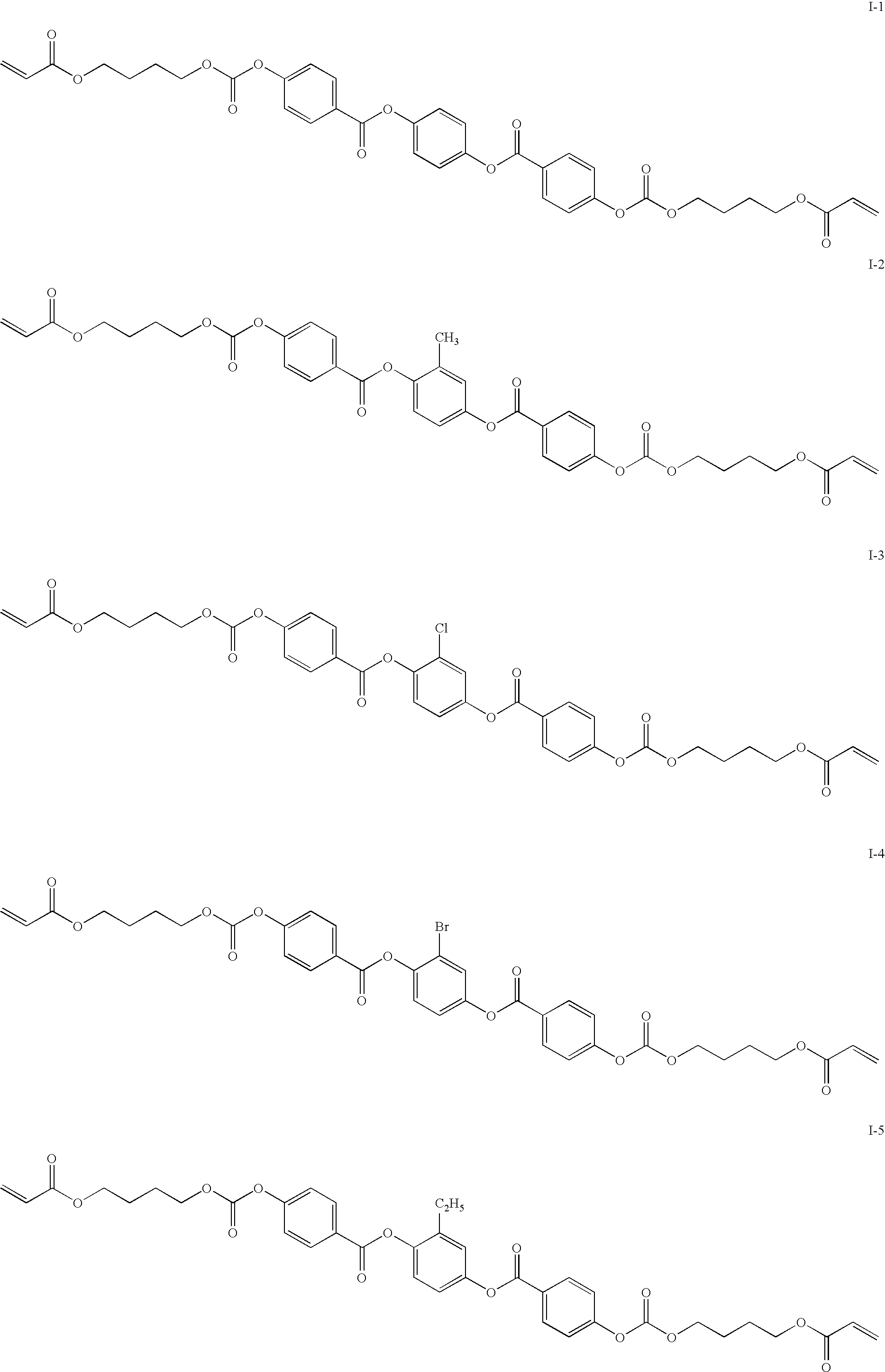

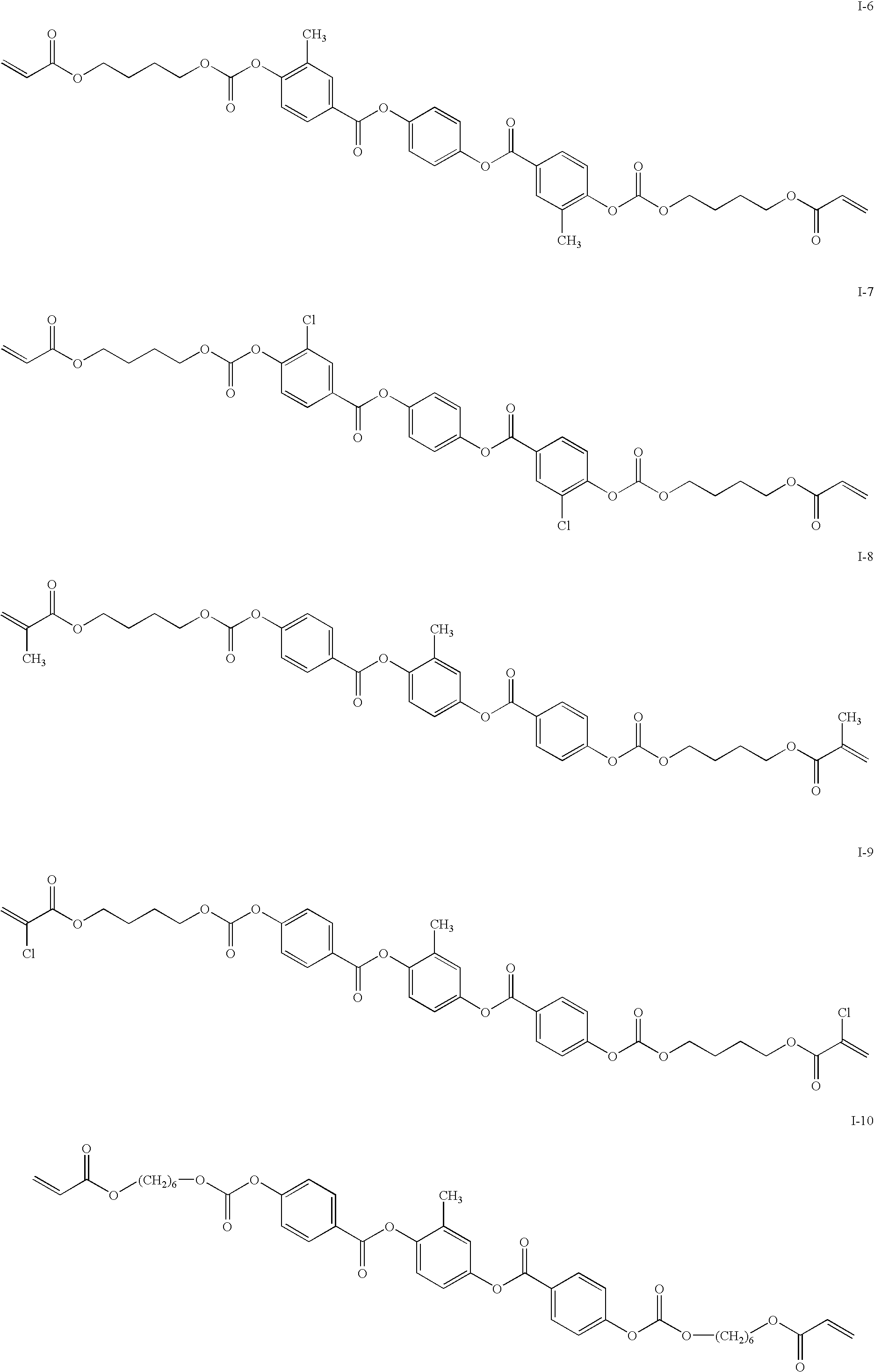

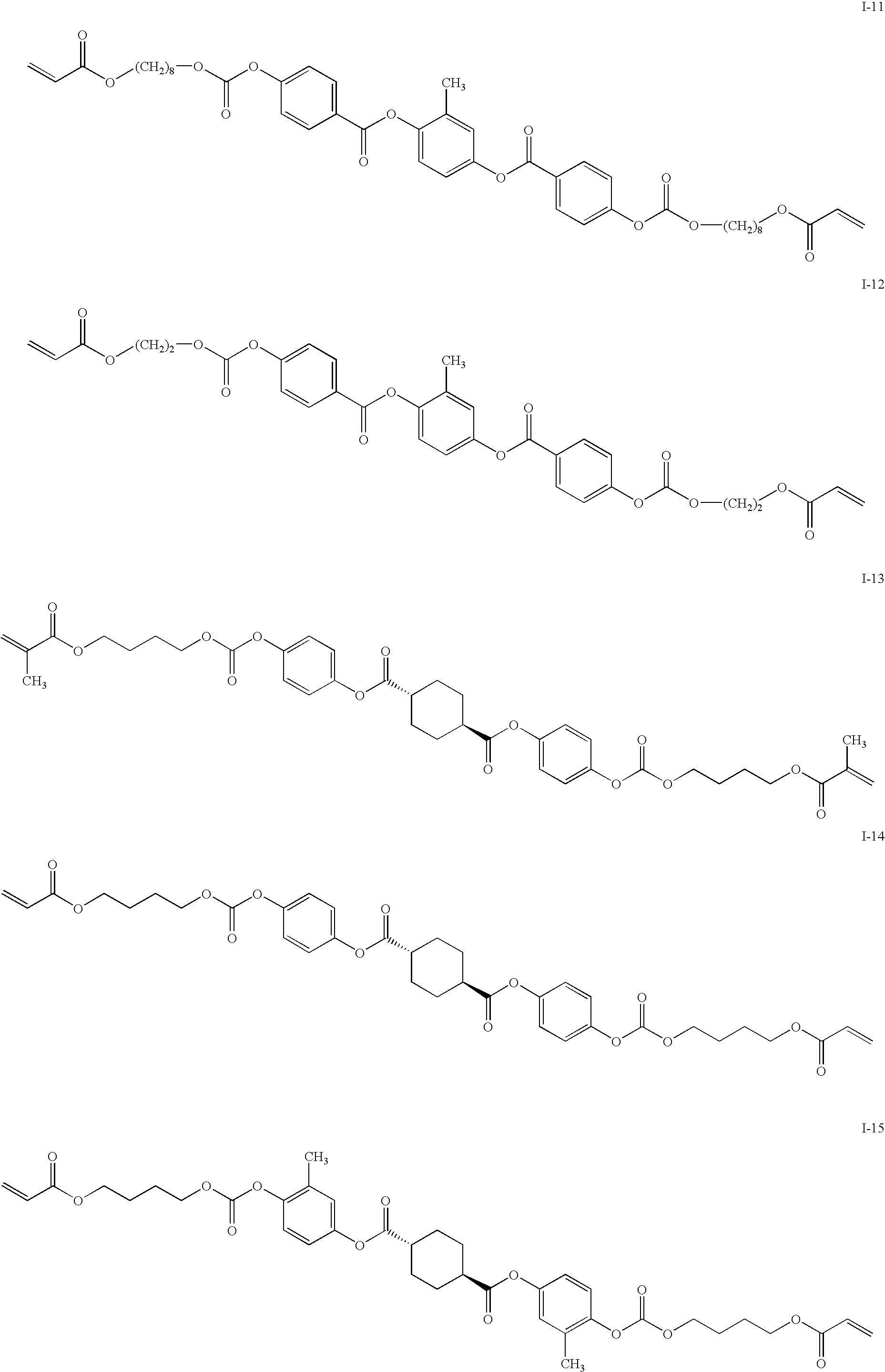

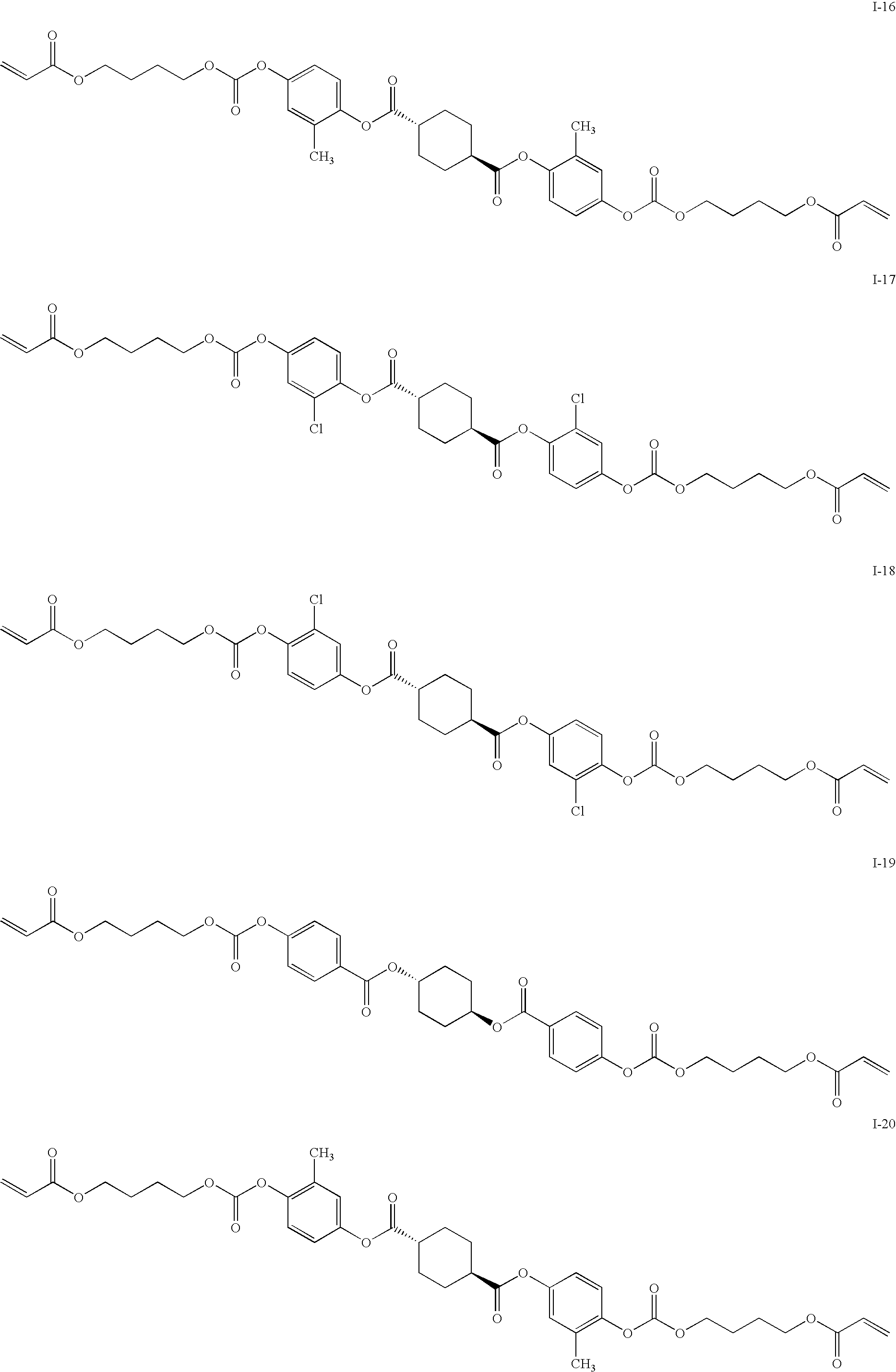

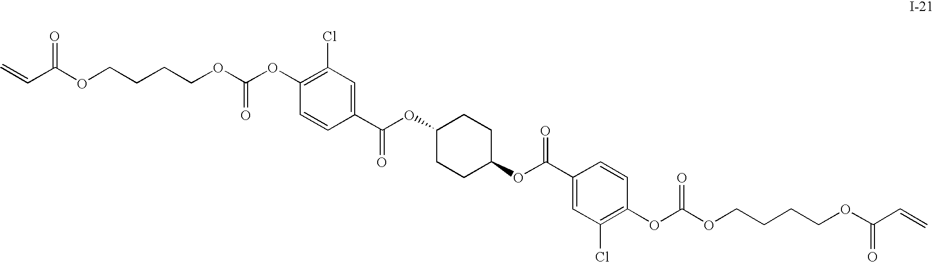

The dichroic polymerization initiator to be used in the step (1) refers to photo-polymerization initiators, especially those having absorption selectivity to a specific direction of polarization, and generating free radicals while being induced by the polarized light. Details and specific examples thereof are described in WO03/05411 A1. Together with the dichroic photo-polymerization initiator, any of conventional polymerization initiators, including α-carbonyl compounds (described in U.S. Pat. Nos. 2,367,661 and 2,367,670), acyloin ether (described in U.S. Pat. No. 2,448,828), aromatic acyloin compounds substituted by α-hydrocarbon (described in U.S. Pat. No. 2,722,512), polynuclear quinone compound (U.S. Pat. Nos. 3,046,127 and 2,951,758), triaryl imidazole dimer combined with p-aminophenylketone (described in U.S. Pat. No. 3,549,367), acrydine and phenazine compounds (described in Japanese Laid-Open Patent Publication No. 60-105667, U.S. Pat. No. 4,239,850) and oxadiazole compounds (described in U.S. Pat. No. 4,212,970), may be used.

The polymerizable composition to be used in the step (1) may comprise any additives besides the above-described polymerizable liquid crystal compound and dichroic polymerization initiator. Examples of the additives include agents which can promote, in the step (2), aligning the polymerizable liquid crystal compound in a first alignment state. For an exemplary case where the polymerizable liquid crystal compound is horizontally aligned in the step (2), a horizontal alignment agent, described below, is preferably added as the additive.

At least one compound represented by a formula (1), (2) or (3), horizontal alignment agent, shown below may be added to the composition used for preparing the optically anisotropic layer, in order to promote aligning the liquid-crystalline molecules horizontally. It is to be noted that the term “horizontal alignment” means that, regarding rod-like liquid-crystal molecules, the molecular long axes thereof and a layer plane are parallel to each other, and, regarding discotic liquid-crystal molecules, the disk-planes of the cores thereof and a layer plane are parallel to each other. However, they are not required to be exactly parallel to each other, and, in the specification, the term “horizontal alignment” should be understood as an alignment state in which molecules are aligned with a tilt angle against a layer plane less than 10 degree. The tilt angle is preferably from 0 to 5 degree, more preferably 0 to 3 degree, much more preferably from 0 to 2 degree, and most preferably from 0 to 1 degree.

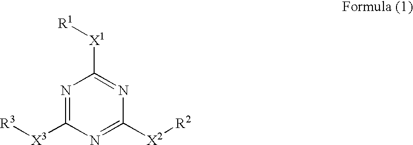

In the formula, R¹, R² and R³ respectively represent a hydrogen atom or a substituent; and X¹, X² and X³ respectively represent a single bond or a divalent linking group. Preferred examples of the substituent represented by R¹ to R³ include an alkyl group (more preferably non-substituted or fluoro-substituted alkyl group), an aryl group (more preferably aryl group having at least one fluoro-substituted alkyl group), a substituted or non-substituted amino, alkoxy and alkylthio, and a halogen atom. The divalent linking group represented by X¹, X² or X³ is preferably selected from the group consisting of an alkylene group, an alkenylene group, a divalent aromatic group, a divalent hetero cyclic residue, —CO—, —NR^{a}— (R^{a} represents a C₁₋₅ alkyl group or a hydrogen atom), —O—, —S—, —SO—, —SO₂— and any combinations thereof. The divalent linking group is more preferably selected from the group consisting of an alkylene group, a phenylene group, —CO—, —NR^{a}—, —O—, —S— —SO₂— and any combinations of at least two selected therefrom. The carbon atom number of the alkylene group is preferably from 1 to 12. The carbon atom number of the alkenylene group is preferably from 2 to 12. the carbon atom numbers of the divalent aromatic group is preferably from 6 to 10.

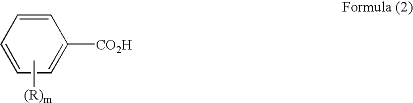

In the formula, R represents a substituent, m is an integer from 0 to 5. When m is 2 or more, plural R are same or different each other. Preferred examples of the substituent represented by R are same as those exemplified above as preferred examples of the substituent represented by R¹, R² and R³. In the formula, m is preferably from 1 to 3, more preferably 2 or 3.

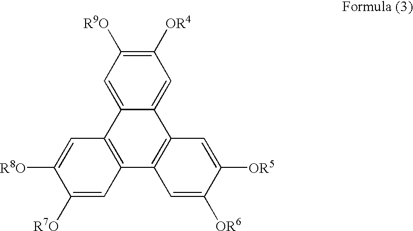

In the formula, R⁴, R⁵, R⁶, R⁷, R⁸ and R⁹ respectively represent a hydrogen atom or a substituent. Preferred examples of the substituent represented by R⁴, R⁵, R⁶, R⁷, R⁸ or R⁹ are same as those exemplified above as preferred examples of the substituent represented by R¹, R² and R³ in the formula (1).

Specific examples of the horizontal alignment agent are similar to those described in Japanese Laid-Open Patent Publication No. 2005-99248, also synthetic methods of which being described in the patent specification.

The polymerizable composition may be prepared as a coating liquid, and a layer formed of this composition may be prepared by applying the coating liquid to the surface of the alignment film and drying it. The solvent used for preparing the coating liquid is preferably selected from organic solvents. Examples of organic solvents include amides (e.g., N,N-dimethyl formamide), sulfoxides (e.g., dimethyl sulfoxide), heterocyclic compounds (e.g., pyridine), hydrocarbons (e.g., benzene, hexane), alkyl halides (e.g., chloroform, dichloromethane), esters (e.g., methyl acetate, butyl acetate), ketones (e.g., acetone, methyl ethyl ketone) and ethers (e.g., tetrahydrofuran, 1,2-dimethoxyethane). Alkyl halides and ketones are preferred. Two or more organic solvents may be used in combination. The process of applying the coating liquid may be carried out according to any known coating method such as extrusion coating, direct gravure coating, reverse gravure coating and die coating while continuous transport is carried out. In the (1) step, two or more layers may be prepared simultaneously, and examples of the method of simultaneous coating are described in U.S. Pat. Nos. 2,761,791, 2,941,898, 3,508,947, 3,526,528, and in “Kotingu Kogaku (Coating Engineering), written by Yuji Harazaki, p. 253, published by Asakura Shoten (1973).

The coating amount is not limited to any range, and it may be decided depending on the preferred thickness of the optically anisotropic layer to be prepared. In general, the thickness of the optically anisotropic layer, to be obtained finally, is preferably from 0.1 to 20 micro meters, and more preferably from 0.5 to 10 micro meters.

The content of the dichroic photo-polymerization initiator in the dichroic polymerizable composition is preferably adjusted to 0.01 to 20% by mass, and more preferably 0.5 to 5% by mass, of the total mass of the composition (total mass of solid content for the case where the composition is prepared in a form of coating liquid). For the case where the compounds expressed by the formulae (1) to (3) are added, the contents thereof are preferably adjusted to 0.01 to 20% by mass, more preferably to 0.01 to 10% by mass, and still more preferably to 0.02 to 1% by mass of the mass of liquid crystal compound. The compounds expressed by the formulae (1) to (3) may be used alone, or in combination of two or more species.

The alignment film to be used in the step (1) is not specifically limited. Preferable examples thereof include an alignment film prepared by rubbing the surface of a polymer layer, an alignment film formed by oblique vacuum evaporation of an inorganic compound, an alignment film having micro-grooves, stacked film (LB film) of ω-tricosanic acid, dioctadecyl dimethyl ammonium chloride, methyl stearate and the like formed by the Langmuir-Blodgett technique, and an alignment film in which dielectric is aligned with the aid of electric field or magnetic field.

The alignment film is preferably prepared by using a polymer. Examples of the polymer include polymers such as polymethyl methacrylate, acrylic acid/methacrylic acid copolymer, styrene/maleimide copolymer, polyvinyl alcohol, poly(N-methylol acrylamide), styrene/vinyltoluene copolymer, chlorosulfonated polyethylene, nitrocellulose, polyvinyl chloride, chlorinated polyolefin, polyester, polyimide, vinyl acetate/vinyl chloride copolymer, ethylene/vinyl acetate copolymer, carboxymethyl cellulose, polyethylene, polypropylene and polycarbonate. The alignment film may be prepared also by using a compound such as silane coupling agent. Preferable examples of the polymer include polyimide, polystyrene, polymer of styrene derivative, gelatin, polyvinyl alcohol, and alkyl-modified polyvinyl alcohol having alkyl groups (preferably having 6 or more carbon atoms).

Species of the polymer applicable herein may be determined depending on alignment of the liquid crystal compound (in particular, mean tilt angle). For example, in the step (2) in the above, any polymers not causative of lowering surface energy of the alignment film (general polymer for alignment) may be used in order to horizontally align the liquid crystal compound. Species of the polymer are specifically described in various literatures relevant to liquid crystal cell or optical compensation film. For example, polyvinyl alcohol or modified polyvinyl alcohol, polyacrylic acid or copolymer of polyacrylic acid ester, polyvinyl pyrrolidone, cellulose or modified cellulose are preferably used. For the purpose of improving the adhesiveness with the resultant optically anisotropic layer, it is also preferable to use a polymer having a polymerizable group. The polymerizable group can be introduced into the polymer in a form of repeating unit having a polymerizable group in the side chain thereof, or in a form of cyclic substituent. It is more preferable to use an alignment film capable of forming chemical bonds at the interface with the liquid crystal compound, wherein examples of such alignment film include those described in Japanese Laid-Open Patent Publication No. H9-152509. Modified polyvinyl alcohols having acryl groups introduced by using acid chloride or Karenz MOI (from Showa Denko KK) are particularly preferable. Thickness of the alignment film is preferably 0.01 to 5 μm, and more preferably 0.05 to 2 μm.

Also polyimide film (preferably fluorine atom-containing polyimide film), widely used as an alignment layer of liquid crystal cells of liquid crystal display devices, may be used as the alignment film. This can be obtained by coating polyamic acid (for example, LQ/LX Series from Hitachi Chemical Co., Ltd., SE series from Nissan Chemical Industries, Ltd., etc.) on the surface of a support, baked at 100 to 300° C. for 0.5 to 1 hour, and rubbed. These polymers may further be cured by introducing reactive groups thereinto, or by using the polymer together with a crosslinking agent such as isocyanate compound or an epoxy compound, and the resultant cured film may be used as the alignment film.

The alignment film is preferably prepared by applying a coating liquid, which is a composition comprising any of the above-described polymers, to the surface to thereby form a polymer layer, and by rubbing the surface of the polymer layer. The coating liquid for an alignment film preferably contains a polymer having reactive groups in the side chains thereof, or monomer or oligomer having functional group(s) which are specifically exemplified by modified polyvinyl alcohol having reactive groups in the side chains thereof. The reactive group is preferably such as being capable of reacting directly with the reactive groups owned by the polymerizable liquid crystal compound used for forming the optically anisotropic layer. By subjecting the polymer in the alignment film and the polymerizable liquid crystal compound in the optically anisotropic layer to direct crosslinking reaction, the obtained film is improved in the strength (adhesiveness between the optically anisotropic layer and the alignment film).

The coating liquid for forming the alignment film may be coated by dip coating, knife coating, air knife coating, curtain coating, roller coating, wire bar coating, gravure coating and extrusion coating (U.S. Pat. No. 2,681,294). Two or more layers may be coated at the same time. Concomitant coating is described in specifications of U.S. Pat. Nos. 2,761,791, 2,941,898, 3,508,947 and 3,526,528, and “Coating Kogaku (Coating Engineering)”, Yuji Harasaki, p. 253, Asakura Shoten (1973).

Processes having widely been adopted to treatment for liquid crystal alignment of LCD may be used for the rubbing. More specifically, an adoptable method is such as rubbing the surface of the alignment layer using paper, gauge, felt, rubber, or nylon or polyester fabric in a certain direction. It is generally accomplished by repeating rubbing several times, typically using cloth piled with fibers of uniform length and thickness with an averaged density. In the present invention, the polymer layer is preferably formed as described in the above, by coating and drying a coating liquid for forming alignment film on the surface of a support being unrolled and fed forward, and then the polymer layer under continuous feeding is rubbed to thereby form the alignment film.

The alignment film is not limited to those composed of the above-described polymer materials, allowing use of inorganic obliquely deposited film or the like. Substances to be deposited in the inorganic obliquely deposited film is primarily represented by SiO₂, and also by metal oxides such as TiO₂ and ZnO₂, fluorides such as MgF₂, and still also by metals such as Au and Al. Any metal oxides may be used as the substances to be obliquely deposited, so far as they have large dielectric constants, without being limited to those described in the above. The inorganic obliquely deposited film may be formed using a vacuum evaporation apparatus. The inorganic obliquely deposited film can be formed by vacuum evaporation onto a fixed film (support) or onto a moving web, and can be used as the alignment film.

The alignment film used in the step (1) may also be an alignment film formed on a support composed of a plastic film or the like. The alignment film may be formed in a continuous manner on the surface of a rolled-up plastic web film being constantly unrolled. The support is not specifically limited, allowing use of various polymer films. Requirements for the support used for producing the optical compensation film of the liquid crystal display device include transparency to the visible light, and optical characteristics thereof not affective to, or conversely contributive to optical compensation. Cellulose acylate film is preferably used. Cellulose acylate adoptable as the support will be described later. For the case where the transfer material described later is produced, materials composing the support are not limited, because the support is separated off after the alignment film is transferred onto a target material for transfer.

[Step (2)]

Next, in the step (2), molecules of the polymerizable liquid crystal compound in the layer formed of the polymerizable composition (referred to as “polymerizable composition layer”, hereinafter) are aligned in a first alignment state. Although there is no need of supplying energy such as by heating, for the case where a desired state of alignment is accomplished in the process of coating and drying of the coating liquid of the polymerizable composition on the surface of the alignment film, the first state of alignment is generally accomplished by heating or cooling depending on the transition temperature of the liquid crystal compound. For the case where the molecules of the polymerizable rod-like liquid crystal compound are aligned in a homogeneous alignment in the step (2), the alignment is generally accomplished by heating at and above room temperature. Preferable temperature range may be determined depending on the transition temperature of the liquid crystal compound. In terms of stabilizing the alignment state, the alignment state is preferably matured, and for this purpose, the layer is preferably allowed to stand in an atmosphere conditioned at a predetermined temperature for a certain time ranging, for example, from 30 seconds to 5 minutes or around. The maturing time is, however, not limited thereto in the continuous producing process, because preferable range of maturing time may vary depending on the feeding time and maturing temperature.

[Step (3)]

Next, in the step (3), the layer formed of the polymerizable composition is irradiated with polarized ultraviolet light, so as to carry out polymerization of the polymerizable liquid crystal compound to proceed, and so as to align molecules of the polymerizable liquid crystal compound in a second alignment state, to thereby form the optically anisotropic layer. Polarized light is irradiated under a condition that the percentage of polarized ultraviolet light having an extinction ratio ranging from 1 to 8 is not greater than 15% with respect to an energy density of polarized ultraviolet light per unit area (J/cm²). In the step (3), radicals are generated from the dichroic polymerization initiator upon irradiation of polarized ultraviolet light, and polymerization of the polymerizable liquid crystal compound proceeds. Because the dichroic polymerization initiator is used, the radicals are generated preferentially in a predetermined direction (generally in parallel) with respect to the direction of polarization of ultraviolet light, rather than being uniformly generated, wherein the polymerization locally proceeds where the radicals were preferentially generated. As a consequence, liquid crystal molecules become to align and be fixed in a second alignment state, which is modified not a little from the first alignment state, and thereby the optically anisotropic layer is formed. It is required for the optically anisotropic layer to satisfy optical characteristics necessary for applications (for example, optical compensation of VA-mode liquid crystal cells), and to possess strength as enough as being durable in the applications thereafter, whereas for highly productive producing, higher speed of feeding in the continuous production, that is, shorter time of irradiation of polarized light, may be more preferable. In the present invention, the optically anisotropic layer having desired optical characteristics and strength can be obtained at an excellent productivity, because the polarized ultraviolet light is irradiated under a condition that the percentage of polarized ultraviolet light having an extinction ratio ranging from 1 to 8 is not greater than 15% with respect to an energy density of polarized ultraviolet light per unit area (J/cm²).

The term “extinction ratio” herein means a ratio of power of planar polarized light beam transmitted through a polarizer, which is disposed on the path of planar polarized light beam to be measured so that the polarization axis of the polarizer is parallel to the light beam plane to power of planar polarized light beam transmitted through a polarizer which is disposed so that the polarization axis of the polarizer is normal to the light beam plane (“Hikari Gijutsu Yogo Jiten” (Terms of Opto-Engineering), 3rd, ed., Shuji Koyanagi, Optronics Co., Ltd.). Although the term generally means a mean value obtained by integrating local energy densities of S-polarized beam and P-polarized beam, or energy density of the entire beam, the term in the present invention means ratio of energy densities of P-polarized beam and S-polarized beam, and further means the ratio having a value of 1 or larger.

The extinction ratio can be determined by ratio of intensity (W/cm²) respectively obtained when an analyzer having transmission axes parallel to or normal to, the polarization axis of a polarizer is disposed at an arbitrary position within the ultraviolet beam plane, between a polarizer polarizing ultraviolet light and the surface of a sample to be irradiated, more preferably closely straight above the surface of a sample to be irradiated and straight under the polarizer.

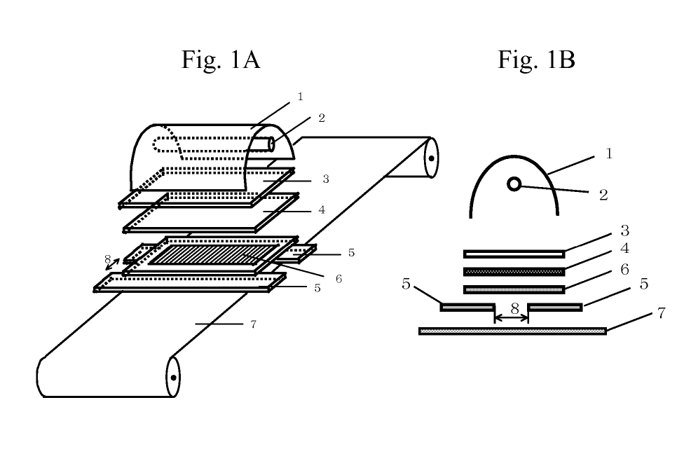

FIGS. 1A and 1B are schematic drawings of a polarized ultraviolet irradiation apparatus applicable to the method of the present invention. FIG. 1A is a schematic perspective view, and FIG. 1B is a schematic sectional view. The polarized ultraviolet irradiation apparatus shown in FIGS. 1A and 1B has a rod-like lamp 2, a reflecting mirror 1 reflecting beam from the lamp 2 into the direction of a surface 7 to be irradiated, an optical component 3 adjusting direction of beam from the lamp 2, a wavelength selection filter 4 adjusting irradiation wavelength, a polarizer 6 separating a polarization component from the beam from the lamp 2 so as to produce polarized beam, and an aperture 5 blocking unnecessary components of the beam.

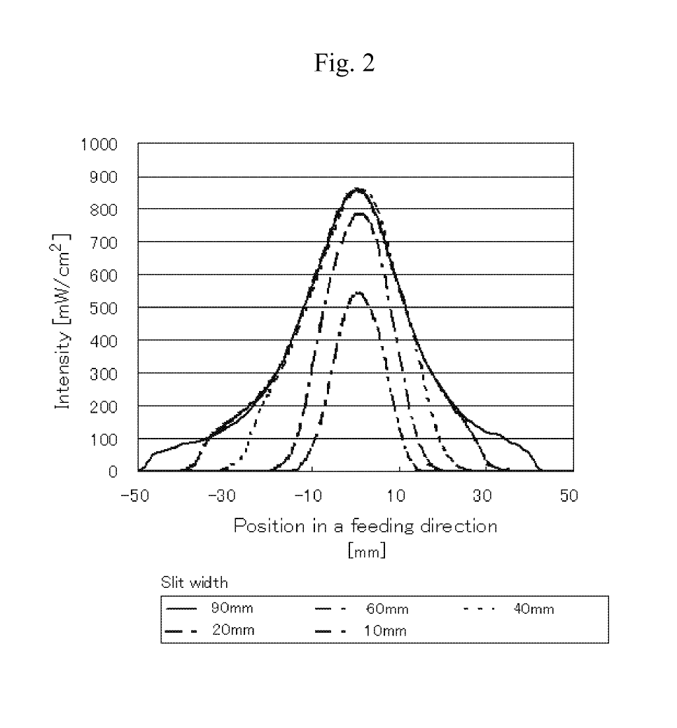

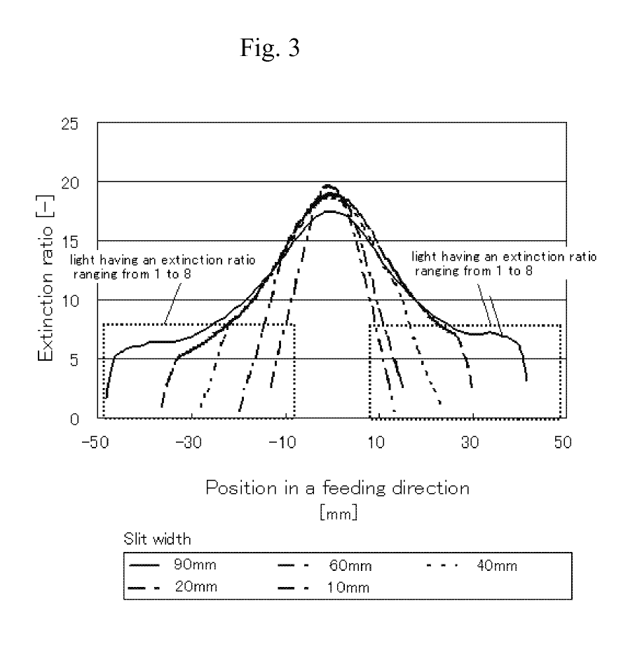

The polarized ultraviolet irradiation apparatus shown in FIGS. 1A and 1B is configured so as to block components having low extinction ratios using the aperture (5), to thereby reduce the percentage of components having extinction ratios ranging from 1 to 8 with respect to a light energy density per unit area. Distribution of intensity 8 mW/cm²) over the surface (7) to be irradiated with respect to the direction of feeding, measured when the slit width (8) of the aperture (5) of the polarized ultraviolet irradiation apparatus shown in FIGS. 1A and 1B was respectively adjusted to 90 mm, 60 mm, 40 mm, 20 mm and 10 mm is shown in FIG. 2, and distribution of extinction ratio with respect to the direction of feeding is shown in FIG. 3. Optical characteristics of light with respect to the direction of feeding are important, because the surface to be irradiated is actually exposed in this way in the continuous production. The measurements were carried out by using an intensity meter “UVPF-A1” from Eyegraphics Co., Ltd. Wire-grid polarizer filters (ProFlux PPL02 (high transmittance type), from Moxtek, Inc.) were used as a polarizer and an analyzer; and a ultraviolet irradiation apparatus based on microwave emission system equipped with a D bulb having an intense emission spectrum at 350 to 400 nm was used as a light source.

It is understandable from the results shown in FIG. 2, that the larger the slit width (8) of the aperture (5) becomes, the higher the intensity (mW/cm²) becomes, showing a tendency that the polymerizable composition layer can be cured within shorter time. On the other hand, as shown in FIG. 3, it is also understandable that widening of the slit width allows irradiation of components having smaller extinction ratios, which raises a tendency that radicals generated from the dichroic polymerization initiator cannot fully be localized. By adjusting the slit width (8) of the aperture (5), the surface is successfully prevented from being irradiated by polarized ultraviolet light having an extinction ratio ranging from 1 to 8 with a percentage greater than 15% with respect to an energy density of polarized ultraviolet light per unit area (J/cm²). As shown in FIG. 3, the percentage of components having smaller extinction ratios increases as a position becomes more distant laterally on both sides from the point on the surface to be irradiated, which falls straight under the lamp (2), so that the slit width (8) is preferably configured as being opened equally on both sides of a line drawn from the center of the lamp (2) towards the surface to be irradiated at right angles.

As the lamp (2) included in the polarized ultraviolet irradiation apparatus, a rod-like lamp such as high-pressure mercury lamp or a metal halide lamp are used. More preferably, the light source is capable of irradiating at least emission beam of 200 nm to 400 nm. Wavelength of the polarized ultraviolet light preferably has a peak at 300 to 450 nm, and more preferably has a peak at 350 to 400 nm. Those excellent in stability of irradiation, large in energy density of light, and long in service life are preferable.

The reflecting mirror (1) included in the polarized ultraviolet irradiation apparatus preferably has large reflectivity to necessary wavelength of ultraviolet radiation. In view of avoiding any deformation or denaturation induced by heat, the mirror 1 preferably has a low reflectivity in the wavelength region of visible light and infrared radiation.

The polarizer (6) included in the ultraviolet irradiation apparatus is not specifically limited so far as it has polarization separation characteristics with respect to ultraviolet radiation of necessary wavelength, allowing use of various products. Absorption-type polarizer can be exemplified by a polarizer obtained by stretching polyvinyl alcohol film doped with iodine or dichroic dye, and a polarizer having dichroic needle crystals aligned therein, and non-absorption-type polarizer can be exemplified by a polarizer adopting Brewster's angle, a polarizer composed of a dielectric multi-layered film, a wire-grid polarizer, and diffusion-type polarizer. The non-absorption-type polarizer is preferable, and among others, a wire grid polarizer having a high polarization separation performance over a wide wavelength region and a high processability and formability processability is particularly preferable.

The wire grid polarizer has conventionally been used in the field of radar handling electric wave, infrared astrological observation instruments and so forth, and has gradually been adopted in the field handling the visible light such as in liquid crystal projector system. The wire grid polarizer is composed of a plurality of parallel electro-conductive electrodes supported by a substrate, and is characterized by pitch or periodicity of the conductors, width of the individual conductors, and thickness of the conductors. Metal wire made of silver, chromium, aluminum or the like is adoptable to the electric conductors of the wire grid polarizer.

The straight electric conductors of the wire grid polarizer may be produced by employing lithographic technique and etching technique. Sectional geometry of the electric conductors is defined by the pitch and the width of the electric conductors, the height of the electric conductors, and the length of the electric conductors, and falls within a range attainable by the lithographic technique and etching technique used for producing the straight electric conductors. As for structure of the wire grid polarizer, preferable ranges of size and configuration are determined depending on the wavelength of light to be irradiated. The length of the electric conductors is good enough if they are longer than the wavelength of polarized light so far as they can be produced. The electric conductors of longer than 100 nm are generally used. The pitch of the electric conductors is preferably not larger than half, and more preferably ⅓, of the wavelength of ultraviolet light to be irradiated, that is, 1.5 to 0.06 μm. The width of the electric conductors is preferably not larger than half, and more preferably ⅓, of the wavelength of ultraviolet radiation to be irradiated, and is more preferably 10% to 90% of the pitch. The height of the electric conductors is preferably 0.005 to 0.5 μm. Range of wavelength over which polarization separation by the wire grid polarizer is attainable depends on the pitch of the electric conductors. More specifically, polarization is attainable without lowering the extinction ratio if the pitch falls in the range approximately from ±50 to ±100 nm of the wavelength of ultraviolet radiation to be polarized, whereas any components of radiation exceeding this range may lower the extinction ratio.

The wavelength selection filter (4) owned by the polarized ultraviolet irradiation apparatus is not specifically limited. Examples of the filter include edge filter and band-pass filter. There is no special limitation also on position of the wavelength selection filter (4), wherein the polarizer (6) is preferably disposed more frontward than the wavelength selection filter (4) (that is, a position more closer to the sample to be irradiated). Characteristics of the edge filter and band-pass filter are generally defined by wavelength dependence of transmittance of the edge filter and the band-pass filter, and are expressed by wavelength (λa) giving a transmittance of 0.005, wavelength (λc) giving a transmittance of 0.5, and wavelength (μp) giving a transmittance of 0.95 or above. For example, if there is a need for exciting radical generation with the aid of near-ultraviolet radiation ranging from 350 nm to 400 nm, it is preferable to set λa at around 340 nm, and λp at around 370 nm. By setting λa and λp within a narrow wavelength range, wavelength selectivity of the optical filter can be improved, and by virtue of such characteristics of the edge filter or the band-pass filter, curing degree of the polymerizable composition layer may be adjustable, or an effect of avoiding denaturation of the material may be obtained by preventing the material from being irradiated by unnecessary components of radiation.

The edge filter and band-pass filter comprise layers differed from each other in the refractive index, so as to cause half-wave retardation of light reflected between the layers, to thereby cancel the incident light based on interference of light. Representative examples of the edge filter and the band-pass filter include those having a plurality of thin films composed of inorganic materials formed on a support. Examples of the inorganic material include fluoride compounds such as AlF₃, BaF₂, CaF₂, Na₃AlF₆, DyF₃, GdF₃, LaF₃, MgF₂, NdF₂, TdF₃, YbF₃, and YF₃; oxide compounds such as SiO₂, SiO, Al₂O₃, HfO₂, ZrO₂, Ta₂O₅, Nb₂O₅, TiO₂, In₂O₃, and WO₃; nitride compounds such as SiON, and Si₃N₄; carbide compounds such as SiC, and B₄C; and mixed oxide compounds such as SiO₂/Al₂O₃, Al₂O₃/Pr₆O₁₁, Al₂O₃/La₂O₃, ZrO₂/Ta₂O₅, ZrO₂/MgO, ZrO₂/Al₂O₃, TiO₂/Pr₆O₁₁, TiO₂/Al₂O₃, and TiO₂/La₂O₃.

The inorganic materials can be formed as films on a support, by vacuum evaporation, electron beam evaporation, ion beam evaporation, plasma evaporation or sputtering. As the support, ozone-less quartz glass, synthetic quartz glass and natural quartz glass, which are excellent in transmissivity to ultraviolet radiation and stable to heat, are preferably used.

The optical component (3) adjusting the direction of light, included in the polarized ultraviolet irradiation apparatus, may be any component capable of changing direction of light. For example, optical lenses (cylindrical lens and collimator lens) and so forth may be adoptable, wherein preferable materials of which include ozone-less quartz glass, synthetic quartz glass and natural quartz glass excellent in transmissivity to ultraviolet radiation and stable to heat. Position of the optical component may be on the lamp side or on the side of the surface to be irradiated with respect to the polarizer, or still may be on the both.

The aperture (5) included in the polarized ultraviolet irradiation apparatus is preferably such as being composed of metal plates, having the surface processed to lower the reflectivity to ultraviolet radiation, disposed while keeping a predetermined slit width (8), or an optical filter capable of absorbing ultraviolet radiation and having a predetermined slit width. Position of the aperture (5) may be on the lamp (2) side or on the side of the surface (7) to be irradiated with respect to the polarizer (6), or still may be on the both.

Configuration of the polarized ultraviolet irradiation apparatus adoptable to the method of the present invention is not limited to that shown in FIGS. 1A and 1B, and may include any other components. For example, a plurality of polarized ultraviolet irradiation apparatuses of the invention may be disposed in the longitudinal direction of the lamp, as being adapted to the width of irradiation, or an additional mechanism of cooling the individual constituents of the polarized ultraviolet irradiation apparatus may be disposed. All components possibly irradiated by ultraviolet light, for example, the aperture (5), the wavelength selection filter (4), the polarizer (6), and the light-adjusting components (the reflecting mirror (1) reflecting light from the lamp (2) towards the surface (7) to be irradiated, and the optical component (3) adjusting the direction of light from the lamp (2)) preferably have the cooling mechanism of their own. Among others, the optical filters such as the polarizer (6) and the wavelength selection filter (4), in particular, preferably have cooling mechanisms, because they may be varied in their performances due to heat generated by ultraviolet irradiation, and may be very likely to shorten their service lives. For the case where there is a substrate or the like supporting the polarizer (6) and the wavelength selection filter (4), the cooling mechanism is composed of an inlet allowing therethrough injection of a coolant and an outlet allowing therethrough discharge of the coolant, opened in the side faces of the substrate, a fluid passageway or a cavity allowing therethrough circulation of the coolant, formed in the substrate, and so forth. Provision of such cooling mechanism can moderate the excessive heating, and degradation as a consequence, of the optical filters such as the polarizer and the wavelength selection filter due to ultraviolet irradiation. It is, therefore, made possible to carry out the film forming process in a more stable manner, and to further improve the productivity. There is no special limitation on the geometry of the passageway of coolant inside the substrate, allowing any geometry such as penetrating the substrate straightly from the inlet to the outlet, or such as having kinked and branched portions, especially for the case where a plurality of polarizers are disposed, so as to ensure efficient cooling of these optical filters. Alternatively, the substrate may have a cavity over the entire inner portion thereof.

The coolant adopted herein is not specifically limited. For the case where the polarizer is disposed below a cooling unit, a liquid coolant adopted herein preferably has a refractive index of 1.30 to 1.60. Similarly, in order to avoid lowering in the irradiation efficiency due to absorption of ultraviolet radiation from the light source by the coolant, the coolant is preferably selected from material showing no absorption at least in the wavelength region of ultraviolet light to be irradiated, and is more preferably selected from those showing substantially no absorption in the ranges from 240 to 780 nm, more preferably from 300 to 700 nm, and still more preferably from 330 to 600 nm. Examples of the coolant having refractive index in the above-described range and showing no absorption in the above-described wavelength ranges include air, pure water, alcohols (e.g., ethylene glycol, propylene glycol, glycerin, methanol, ethanol, isopropyl alcohol), and silicone oil. Mixed solution of pure water and alcohols is also preferable.

Temperature of the coolant is preferably 10 to 70° C., more preferably 15 to 60° C., and still more preferably 20 to 50° C.

The polarized ultraviolet irradiation apparatus may further comprise, for example, a conveyor means conveying the web-form support (polymer film, for example) having formed thereon the polymerizable composition layer irradiated by the polarized ultraviolet light, or a support component supporting the polarizer as being freely movable so as to allow irradiation of ultraviolet light without being mediated by the polarizer, or an additional light source capable of irradiating a sample to be irradiated with ultraviolet light or the like, without being mediated by the polarizer, disposed on the more upstream side or more downstream side in the direction of sample feeding.

In the step (3), an energy density of polarized ultraviolet light is preferably 100 mJ/cm² to 5 J/cm², more preferably 150 mJ/cm² to 3 J/cm², and still more preferably 200 mJ/cm² to 2 J/cm². The energy density is determined depending on intensity of the light source used for irradiating polarized ultraviolet light, and irradiation time. The intensity is preferably 20 mW/cm² to 2000 mW/cm², more preferably 100 mW/cm² to 1500 mW/cm², and still more preferably 200 mW/cm² to 1000 mW/cm². As described in the above, for the case where components having lower extinction ratios are blocked by the aperture, narrowing of the slit width gives better results but concomitantly lowers the intensity. In order to ensure an appropriate level of energy density, despite some lowering in the intensity caused by narrowing the slit width, it is preferable to adjust the irradiation time. For the case of continuous irradiation while feeding the support, the irradiation time may be adjustable within a preferable range, by controlling the feeding speed. For an exemplary case with a slit width of 20 to 90 mm, the feeding speed is preferably adjusted within the range from 1 m/min to 10 m/min, and more preferably from 1 m/min to 5 m/min. The feeding speed is, however, not limited to the above-described ranges, because the above-descried appropriate energy density may sometimes be obtained even under a small slit width, if the number of light source is increased or if position of the light source is adjusted.

For the case where components of light having smaller extinction ratios are blocked using the aperture, the percentage of polarized ultraviolet light having extinction ratio ranging from 1 to 8 is preferably exceeds 5% but not greater than 15%, and more preferably 7% to 15%, with respect to the energy density of polarized ultraviolet light, in terms of keeping a desirable level of productivity (that is, in terms of keeping an appropriate level of feeding speed).

In the step (3), the polarized ultraviolet light may be irradiated under heating. The polarized ultraviolet light is preferably irradiated, while keeping the temperature of the surface of the polymerizable composition layer at around the isotropic phase transition temperature Tᵢₛₒ of the polymerizable liquid crystal compound. More specifically, the surface temperature of the polymerizable composition layer during irradiation of the polarized ultraviolet light is preferably kept at Tᵢₛₒ−50 to Tᵢₛₒ° C., and more preferably at Tᵢₛₒ−30 to Tᵢₛₒ° C. By adjusting the surface temperature within the above-described ranges, disturbance of alignment can be moderated as compared with the case where the polarized ultraviolet light is irradiated at temperatures lower than the above-described ranges, and thereby the surface condition of the resultant optically anisotropic layer can be improved. The surface temperature can be measured using an infrared radiation thermometer (for example, IT2-01 from Keyence Corporation).

In order to adjust the surface temperature to the above-described ranges, heating is preferably effected over a period ranging from 10 seconds earlier than the start of irradiation of the polarized ultraviolet light up to 300 seconds after the start of irradiation of the polarized ultraviolet light, and more preferably over a period ranging from 10 seconds earlier than the start of irradiation of the polarized ultraviolet light up to 10 seconds after the start of irradiation of the polarized ultraviolet light. Too short duration of time keeping the surface temperature at the above-described range will fail in promoting the film-forming reaction of the polymerizable composition, also raising a producing-related problem such as expansion of the facility.

There is no special limitation on the method of heating, wherein it is preferable to blow an oxygen shielding gas conditioned at the above-described temperature range into a zone, in the process of irradiating the polarized ultraviolet light, and in the process of feeding and post-heating carried out by requests. It is also possible to adopt, in combination with or in place of the injection, a method of contacting a polymer film, a support, with a heated roll, a method of blowing hot nitrogen, a method of irradiating far-infrared radiation or infrared radiation. A method of heating by supplying warm water or steam to a rotating metal roll, described in Japanese Patent No. 2523574, is also applicable.

According to the invention, the optically anisotropic layer contains a liquid crystal compound, and for the case where in-plane distribution of the liquid crystal or irregularity of the alignment film directly affects the optical characteristics, it is necessary to keep a constant temperature distribution of the entire film including the support in the thickness-wise direction. A method of blowing hot nitrogen, or a method of irradiating far-infrared radiation or infrared radiation may preferably be used. For the case where temperature of the film is controlled through contact with the heated roll as described in the above, it is necessary to keep the temperature distribution in the thickness-wise direction of the film, which may effectively be accomplished in combination with the method of blowing hot nitrogen.

In consideration of strength of the resultant optically anisotropic layer, polarized ultraviolet light is preferably irradiated in the step (3) under an atmosphere having an oxygen concentration of 3% by volume or below, more preferably 1% by volume or below, and still more preferably 0.5% by volume or below. Irradiation of the polarized ultraviolet light in an inert gas atmosphere having an oxygen concentration adjusted to the above-described ranges is preferable in terms of strength of the resultant optically anisotropic layer. Oxygen concentration in the atmosphere of heating before irradiation of the polarized ultraviolet light, and of heating after the curing optionally carried out is preferably adjusted to 10% by volume or below, more preferably 5% by volume or below, still more preferably 3% by volume or below, and even more preferably 1% by volume or below. Means for lowering the oxygen concentration relates to a method of substituting the air (nitrogen concentration of ca. 79% by volume, and oxygen concentration of ca. 21% by volume) with other inert gas. Examples of the inert gas include chemically inactive gases (helium, argon, nitrogen), fron, carbon dioxide gas and so forth listed in Annexed List 6 of the Ordinance on Prevention of Oxygen Deficiency. In particular, nitrogen is preferably used by virtue of its chemical stability and inexpensiveness.

In order to keep the oxygen concentration within the above-described ranges, and to keep a predetermined temperature, it is preferable to inject an oxygen shielding gas conditioned at a predetermined temperature (preferably 40° C. or above) into the zone, in the process of curing and/or feeding. It is still also possible to discharge the inert gas, used for lowering the oxygen concentration in the zone where the processes of curing and/or feeding take place, into a preceding lower oxygen concentration zone and/or a succeeding zone for feeding. This configuration is preferable in view of effectively using the inert gas and saving costs for the producing.

One example of the optically anisotropic layer formed after the step (3) is so-called biaxial optical anisotropic layer, characterized in that the retardation value measured by making incidence of beam of λ nm in the direction inclined +40° away from the normal line on the optical compensation film while assuming the in-plane slow axis as the axis of inclination (axis of rotation), and retardation value measured by making incidence of beam of λnm in the direction inclined −40° away from the normal line on the optical compensation film while assuming the in-plane slow axis as the axis of inclination (rotation axis) are adjusted substantially equal to each other. This sort of optically anisotropic layer can be prepared by using a polymerizable rod-like liquid crystal compound, allowing the rod-like liquid crystal molecules to align in the step (2) so as to produce cholesteric alignment or hybridized cholesteric alignment having the molecules twisted while being gradually varied in the tilt angles in the thickness-wise direction (first alignment state), and in the step (3), by irradiating polarized ultraviolet light so as to produce and localize radicals from the dichroic polymerization initiator, and to proceed polymerization, to thereby distort the cholesteric or hybridized cholesteric alignment (second alignment state). The dichroic polymerization initiator contributive to distortion of the alignment with the aid of polarized ultraviolet light is described in International Patent WO03/054111.

Re of the biaxial optically anisotropic layer formed in the step (3) according to the method of the present invention is preferably from 5 to 250 nm, more preferably from 10 to 100 nm, and still more preferably from 20 to 80 nm. Rth is preferably from 30 to 500 nm as being totaled with Rth of the transparent support, more preferably from 40 to 400 nm, and much more preferably from 100 to 350 nm.

The biaxial optically anisotropic layer having these optical characteristics is particularly used for optical compensation of VA-mode liquid crystal display devices.

[(4) Step of Light Irradiation for Post-Treatment]

In order to further enhance adhesiveness between the optically anisotropic layer and the alignment film, and strength of the optically anisotropic layer, polarized or non-polarized ultraviolet light may further be irradiated (referred to as “(4) step of light irradiation for post-treatment”, hereinafter). Ultraviolet light used for light exposure for the post-treatment may be polarized or may be non-polarized, wherein non-polarized light is preferable in terms of obtaining a large energy density. The irradiation for post-treatment may adopt polarized light alone, or polarized light combined with non-polarized light, wherein for the case of combination, irradiation of polarized light preferably precedes irradiation of non-polarized light. The ultraviolet irradiation may be carried out without substitution with an inert gas, but is preferably carried out in an inert gas atmosphere having an oxygen concentration 0.5% or below. The energy density of ultraviolet light to be used for post-treatment is preferably from 20 mJ/cm² to 10 J/cm², and more preferably from 20 to 300 mJ/cm². Intensity is preferably 20 to 1200 mW/cm², more preferably 50 to 1000 mW/cm², and still more preferably 100 to 800 mW/cm². Wavelength of irradiation, for the case of irradiation of polarized light, is preferably such as having a peak in the range from 300 to 450 nm, more preferably from 350 to 400 nm. For the case of irradiation of non-polarized light, wavelength of irradiation is preferably such as having a peak in the range from 200 to 450 nm, and more preferably 250 to 400 nm.

The optical film produced by going through the step (3), or by further optionally going through the step (4), may directly be incorporated, as the optical compensation film or the like, into the liquid crystal display device. It is also possible to prepare the alignment film while continuously feeding the web-form polymer film, to carry out the steps (1) to (3), and optionally to step (4), and to once roll up the resultant web-form optical film. Thereafter, the film may be put into practical use, after being cut typically according to size of the liquid crystal display device.

The film may be given in a form of polarizer plate having an optically anisotropic layer formed thereon, after being undergone through the step (5) for stacking the polarizer film as described below, or may be given in a form of transfer material, after being undergone through the step (6) for forming a photosensitive polymer layer as described below.

[(5) Step of Stacking Polarizer Film]

As described in the above, the polarizer plate may be produced by bonding, in a roll-to-roll manner, three films which are the web-form optical film once rolled up, the web-form polarizer film similarly once roller up, and a polymer film for protection. The roll-to-roll stacking is preferable not only in terms of productivity, but also because the polarizer plate is less causative of dimensional change or curling, and can be imparted with an excellent mechanical stability. The back surface (surface having no optically anisotropic layer formed thereon) to be bonded with the polarizer film, and the surface of the polymer film for protection may be saponified. An adhesive may preferably used for the bonding, wherein a polyvinyl alcohol-base adhesive, which is the same material with the polarizer film, is preferable in general.

Of course, the polarizer plate may be produced by cutting each of three films into a predetermined size, and then by stacking these films.

[(6) Step of Forming Photosensitive Polymer Layer]

A transfer material can be produced by preparing a photosensitive polymer layer on the optically anisotropic layer prepared by going through the step (3), or by further optionally going through the step (4). This sort of transfer material is particularly useful when the optically anisotropic layer is transferred onto a substrate to be transferred, for later patterning for producing a desired pattern. For example, it is particularly useful for the case where the optically anisotropic layer is transferred onto a substrate for a liquid crystal cell, and optical compensation is optimized by partitioning the optically anisotropic layer into domains corresponding to R, G and B subpixels.

The photosensitive polymer layer may be prepared by applying a coating liquid, which is a photosensitive polymer composition, to the surface of the optically anisotropic layer and drying it. The photosensitive polymer composition may be positive or negative type. One examples of the photosensitive polymer composition is a polymer composition comprising (1) an alkaline-soluble polymer, (2) a monomer or oligomer, and (3) a photopolymerization initiator or photopolymerization initiator system. In an embodiment in which the optically anisotropic layer is formed on the substrate at the same time with the photosensitive polymer layer to be used as a color filter, it is preferable to use a colored polymer composition additionally comprising (4) a colorant such as dye or pigment.

These components (1) to (4) will be explained below.

(1) Alkali-Soluble Polymer

The alkali-soluble polymer (which may be referred simply to as “binder”, hereinafter) is preferably a polymer having, in the side chain thereof, a polar group such as carboxylic acid groups or carboxylic salt. Examples thereof include methacrylic acid copolymer, acrylic acid copolymer, itaconic acid copolymer, crotonic acid copolymer, maleic acid copolymer, and partially-esterified maleic acid copolymer described in Japanese Laid-Open Patent Publication “Tokkaisho” No. 59-44615, Examined Japanese Patent Publication “Tokkosho” Nos. 54-34327, 58-12577 and 54-25957, Japanese Laid-Open Patent Publication “Tokkaisho” Nos. 59-53836 and 59-71048. Cellulose derivatives having on the side chain thereof a carboxylic acid group can also be exemplified. Besides these, also cyclic acid anhydride adduct of hydroxyl-group-containing polymer are preferably used. Particularly preferable examples include copolymer of benzyl(meth)acrylate and (meth)acrylic acid described in U.S. Pat. No. 4,139,391, and multi-system copolymer of benzyl(meth)acrylate and (meth)acrylic acid and other monomer. These binder polymers having polar groups may be used independently or in a form of composition comprising a general film-forming polymer. The content of the polymer generally falls in the range from 20 to 50% by mass, and more preferably from 25 to 45% by mass, of the total weight of the solid components contained in the polymer composition.

(2) Monomer or Oligomer

The monomer or oligomer used for the photosensitive polymer layer is preferably selected from compounds, having two or more ethylenic unsaturated double bonds, capable of causing addition polymerization upon being irradiated by light. As such monomer and oligomer, compounds having at least one ethylenic unsaturated group capable of addition polymerization, and having a boiling point of 100° C. or above under normal pressure can be exemplified. The examples include monofunctional acrylates and monofunctional methacrylates such as polyethylene glycol mono(meth)acrylate, polypropylene glycol mono(meth)acrylate and phenoxyethyl(meth)acrylate; multi-functional acrylate and multi-functional methacrylate, obtained by adding ethylene oxide or propylene oxide to multi-functional alcohols such as trimethylol propane and glycerin, and then converting them into (meth)acrylates, such as polyethylene glycol di(meth)acrylate, polypropylene glycol di(meth)acrylate, trimethylolethane triacrylate, trimethylolpropane tri(meth)acrylate, trimethylolpropane diacrylate, neopentyl glycol di(meth)acrylate, pentaerythritol tetra(meth)acrylate, pentaerythritol tri(meth)acrylate, dipentaerythritol hexa(meth)acrylate, dipentaerythritol penta(meth)acrylate, hexanediol di(meth)acrylate, trimethylol propane tri(acryloyloxypropyl)ether, tri(acryloyloxyethyl)isocyanurate, tri(acryloyloxyethyl)cyanurate, glycerin tri(meth)acrylate.

Additional examples of multi-functional acrylates and methacrylates include urethane acrylates such as those described in Examined Japanese Patent Publication “Tokkosho” Nos. 48-41708, 50-6034 and Japanese Laid-Open Patent Publication “Tokkaisho” No. 51-37193; polyester acrylates such as those described in Japanese Laid-Open Patent Publication “Tokkaisho” No. 48-64183, Examined Japanese Patent Publication “Tokkosho” Nos. 49-43191 and 52-30490; and epoxyacrylates which are reaction products of epoxy polymer and (meth)acrylic acid. Of these, trimethylolpropane tri(meth)acrylate, pentaerythritol tetra(meth)acrylate, dipentaerythritol hexa(meth)acrylate, dipentaerythritol penta(meth)acrylate are preferable.

Besides these, also “polymerizable compound B” described in the Japanese Laid-Open Patent Publication “Tokkaihei” No. 11-133600 are exemplified as the preferable examples.

These monomers or oligomers can be used independently or in combination of two or more species thereof. The content of the monomer or oligomer generally falls in the range from 5 to 50% by mass, and more preferably from 10 to 40% by mass, of the total weight of the solid components contained in the polymer composition.

The monomer or oligomer to be used for preparing the photosensitive polymer layer may be selected from cation-polymerizable monomers and oligomers. Examples of such monomer and oligomer include epoxy-base compounds such as cyclic ethers, cyclic formals, acetals, vinylalkyl ethers and compounds having a thirane group bisphenol-type epoxy resins, novolac-type epoxy resins, alicyclic epoxy resins, epoxidized unsaturated fatty acids and epoxidized polybutadienes. More specific examples of such monomer and oligomer include compounds described in “New Epoxy Resins (Shin Epokishi Jushi)” written and edited by Hiroshi Kakiuchi (published by SHOKODO CO., LTD in 1985) and “Epoxy Resins (Epokishi Jushi)” written and edited by Kuniyuki Hashimoto (published by NIKKAN KOGYO SHIMBUN, LTD in 1969); and 3-functional glycidyl ethers (e.g., trimethylolethane triglycidyl ether, trimethylolpropane triglycidyl ether, glycerol triglycidyl ether, triglycidyl tris hydroxy ethylisocyanurate), 4- or more functional glycidyl ethers (e.g., sorbitol tetraglycidyl ether, pentaerythritol tetraglycidyl ether, polyglycidyl ether of cresol-novolac resin and polyglycidyl ether of phenol-novolac resin), 3- or more functional alicyclic epoxys (e.g., “EPOLEAD GT-301”, “EPOLEAD GT-401” and “EHPE”, all of which are available from DAICEL CHEMICALINDUSTRIES, LTD., and polycyclohexyl epoxymethyl ether of phelol-novolac resin), and 3- or more functional oxetanes (e.g., “OX-SQ” and “PNOX-1009”, all of which are available from TOAGOSEI CO., LTD.).

(3) Photopolymerization Initiator or Photopolymerization Initiator System

The photopolymerization initiator or photopolymerization initiator system used for the photosensitive polymer layer can be exemplified by vicinal polyketaldonyl compounds disclosed in U.S. Pat. No. 2,367,660, acyloin ether compounds described in U.S. Pat. No. 2,448,828, aromatic acyloin compounds substituted by α-hydrocarbon described in U.S. Pat. No. 2,722,512, polynuclear quinone compounds described in U.S. Pat. Nos. 3,046,127 and 2,951,758, combination of triaryl imidazole dimer and p-aminoketone described in U.S. Pat. No. 3,549,367, benzothiazole compounds and trihalomethyl-s-triazine compounds described in Examined Japanese Patent Publication “Tokkosho” No. 51-48516, trihalomethyl-triazine compounds described in U.S. Pat. No. 4,239,850, and trihalomethyl oxadiazole compounds described in U.S. Pat. No. 4,212,976. Trihalomethyl-s-triazine, trihalomethyl oxadiazole and triaryl imidazole dimer are particularly preferable.

Besides these, “polymerization initiator C” described in Japanese Laid-Open Patent Publication “Tokkaihei” No. 11-133600 can also be exemplified as a preferable example. The content of the photopolymerization initiator or the photopolymerization initiator system generally falls in the range from 0.5 to 20% by mass, and more preferably from 1 to 15% by mass, of the total weight of the solid components of the photosensitive polymer composition.

(4) Colorant

The photosensitive polymer composition may be added with any of known colorants (dyes, pigments). The pigment is desirably selected from known pigments capable of uniformly dispersing in the photosensitive polymer composition, and that the grain size is adjusted to 0.1 μm or smaller, and in particular 0.08 μm or smaller.

The known dyes and pigments can be exemplified by pigments and so forth described in paragraph [0033] in Japanese Laid-Open Patent Publication “Tokkai” No. 2004-302015 and in column 14 of U.S. Pat. No. 6,790,568.

Of the above-described colorants, those preferably used in the present invention include (i) C.I. Pigment Red 254 for the colored polymer composition for R(red), (ii) C.I. Pigment Green 36 for the colored polymer composition for G(green), and (iii) C.I. Pigment Blue 15:6 for the colored polymer composition for B(blue). The above-described pigments may be used in combination.

Preferable examples of combination of the above-described pigments include combinations of C.I. Pigment Red 254 with C.I. Pigment Red 177, C.I. Pigment Red 224, C.I. Pigment Yellow 139 or with C.I. Pigment Violet 23; combinations of C.I. Pigment Green 36 with C.I. Pigment Yellow 150, C.I. Pigment Yellow 139, C.I. Pigment Yellow 185, C.I. Pigment Yellow 138 or with C.I. Pigment Yellow 180; and combinations of C.I. Pigment Blue 15:6 with C.I. Pigment Violet 23 or with C.I. Pigment Blue 60.

Contents of C.I. Pigment Red 254, C.I. Pigment Green 36 and C.I. Pigment Blue 15:6 in the combined pigments are preferably 80% by mass or more, and particularly preferably 90% by mass or more for C.I. Pigment Red 254; preferably 50% by mass or more, and particularly preferably 60% by mass or more for C.I. Pigment Green 36; and 80% by mass or more, and particularly preferably 90% by mass or more for C.I. Pigment Blue 15:6.

The pigments are preferably used in a form of dispersion liquid. The dispersion liquid may be prepared by adding a composition, preliminarily prepared by mixing the pigment and a pigment dispersant, to an organic solvent (or vehicle) described later for dispersion. The vehicle herein refers to a portion of medium allowing the pigments to disperse therein when the coating material is in a liquid state, and includes a liquidous portion (binder) binding with the pigment to thereby solidify a coated layerwand a component (organic solvent) dissolving and diluting the liquidous portion. There is no special limitation on dispersion machine used for dispersing the pigment, and any known dispersers described in “Ganryo no Jiten (A Cyclopedia of Pigments)”, First Edition, written by Kunizo Asakura, published by Asakura Shoten, 2000, p. 438, such as kneader, roll mill, attoritor, super mill, dissolver, homomixer, sand mill and the like, are applicable. It is also possible to finely grind the pigment based on frictional force, making use of mechanical grinding described on p. 310 of the same literature.

The colorant (pigment) used in the present invention preferably has a number-averaged grain size of 0.001 to 0.1 μm, and more preferably 0.01 to 0.08 μm. A number-averaged grain size of less than 0.001 μm makes the pigment more likely to coagulate due to increased surface energy, makes the dispersion difficult, and also makes it difficult to keep the dispersion state stable. A number-averaged grain size exceeding 0.1 μm undesirably causes pigment-induced canceling of polarization, and degrades the contrast. It is to be noted that the “grain size” herein means the diameter of a circle having an area equivalent to that of the grain observed under an electron microscope, and that the “number-averaged grain size” means an average value of such grain sizes obtained from 100 grains.

The photosensitive polymer layer may be used as a color filter after transferring, and in such a case, the photosensitive polymer layer preferably comprises an appropriate surfactant, in terms of effectively preventing non-uniformity in display (non-uniformity in color due to variation in the film thickness). Any surfactants are applicable so far as they are miscible with the photosensitive polymer composition. Surfactants preferably applicable to the present invention include those disclosed in paragraphs [0090] to [0091] in Japanese Laid-Open Patent Publication “Tokkai” No. 2003-337424, paragraphs [0092] to [0093] in Japanese Laid-Open Patent Publication “Tokkai” No. 2003-177522, paragraphs [0094] to [0095] in Japanese Laid-Open Patent Publication “Tokkai” No. 2003-177523, paragraphs [0096] to [0097] in Japanese Laid-Open Patent Publication “Tokkai” No. 2003-177521, paragraphs [0098] to [0099] in Japanese Laid-Open Patent Publication “Tokkai” No. 2003-177519, paragraphs [0100] to [0101] in Japanese Laid-Open Patent Publication “Tokkai” No. 2003-177520, paragraphs [0102] to [0103] in Japanese Laid-Open Patent Publication “Tokkaihei” No. 11-133600 and those disclosed as the invention in Japanese Laid-Open Patent Publication “Tokkaihei” No. 6-16684. In view of obtaining more larger effects, it is preferable to use any of fluorine-containing surfactants and/or silicon-base surfactants (fluorine-containing surfactant, or, silicon-base surfactant, and surfactant containing both of fluorine atom and silicon atom), or two or more surfactants selected therefrom, wherein the fluorine-containing surfactant is most preferable. For the case where the fluorine-containing surfactant is used, the number of fluorine atoms contained in the fluorine-containing substituents in one surfactant molecule is preferably 1 to 38, more preferably 5 to 25, and most preferably 7 to 20. Too large number of fluorine atoms is undesirable in terms of degrading solubility in general fluorine-free solvents. Too small number of fluorine atoms is undesirable in terms of failing in obtaining effects of improving the non-uniformity.

Particularly preferable surfactants can be those containing a copolymer comprising the units derived from the monomers represented by the formulae (a) and (b) below, having a ratio of mass of formula (a)/formula (b) of 20/80 to 60/40:

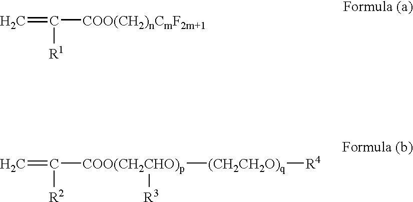

R¹, R² and R³ independently represent a hydrogen atom or a methyl group, R⁴ represents a hydrogen atom or an alkyl group having the number of carbon atoms of 1 to 5. n represents an integer from 1 to 18, and m represents an integer from 2 to 14. p and q represents integers from 0 to 18, excluding the case where both of p and q are 0.

It is to be defined now that a monomer represented by the formula (a) and a monomer represented by the formula (b) of the particularly preferable surfactants are denoted as monomer (a) and monomer (b), respectively. CₘF₂ₘ₊₁ appears in the formula (a) may be straight-chained or branched. m represents an integer from 2 to 14, and is preferably an integer from 4 to 12. Content of CₘF₂ₘ₊₁ is preferably 20 to 70% by mass, and more preferably 40 to 60% by mass, of the monomer (a). R¹ represents a hydrogen atom or a methyl group. n represents 1 to 18, and more preferably 2 to 10. R² and R³ appear in the formula (b) independently represent a hydrogen atom or a methyl group, and R⁴ represents a hydrogen atom or an alkyl group having the number of carbon atoms of 1 to 5. p and q respectively represent integers of 0 to 18, excluding the case where both of p and q are 0. p and q are preferably 2 to 8.

The monomer (a) included in one particularly preferable surfactant molecule may be those having the same structure, or having structures differing within the above-defined range. The same will apply also to the monomer (b).

The weight-average molecular weight Mw of a particularly preferable surfactant preferably falls in the range from 1000 to 40000, and more preferably from 5000 to 20000. The surfactant characteristically contains a copolymer composed of the monomers expressed by the formula (a) and the formula (b), and having a ratio of mass of monomer (a)/monomer (b) of 20/80 to 60/40. Hundred parts by mass of a particularly preferable surfactant is preferably composed of 20 to 60 parts by mass of the monomer (a), 80 to 40 parts by mass of the monomer (b), and residual parts by mass of other arbitrary monomers, and more preferably 25 to 60 parts by mass of the monomer (a), 60 to 40 parts by mass of the monomer (b), and residual parts by mass of other arbitrary monomer.

Copolymerizable monomers other than the monomers (a) and (b) include styrene and derivatives or substituted compounds thereof including styrene, vinyltoluene, α-methylstyrene, 2-methylstyrene, chlorostyrene, vinylbenzoic acid, sodium vinylbenzene sulfonate, and aminostyrene; dienes such as butadiene and isoprene; and vinyl-base monomers such as acrylonitrile, vinylethers, methacrylic acid, acrylic acid, itaconic acid, crotonic acid, maleic acid, partially esterified maleic acid, styrene sulfonic acid, maleic anhydride, cinnamic acid, vinyl chloride and vinyl acetate.

A particularly preferable surfactant is a copolymer of the monomer (a), monomer (b) and so forth, allowing monomer sequence of random or ordered, such as forming a block or graft, while being not specifically limited. A particularly preferable surfactant can use two or more monomers differing in the molecular structure and/or monomer composition in a mixed manner.

Content of the surfactant is preferably adjusted to 0.01 to 10% by mass to the total amount of solid components of the photosensitive polymer layer, and more preferably to 0.1 to 7% by mass. The surfactant is such as containing predetermined amounts of a surfactant of a specific structure, ethylene oxide group and polypropylene oxide group, wherein addition thereof to an amount within a specific range to the photosensitive polymer layer makes it possible to improve non-uniformity in the display on the liquid crystal display device provided with the photosensitive polymer layer as a color filter.

Specific examples of preferred fluorine-containing surfactant include the compounds described in paragraphs [0054] to [0063] of Japanese Laid-Open Patent Publication “Tokkai” No. 2004-163610. It is also possible to directly adopt the commercial surfactants listed below. Applicable commercial surfactants include fluorine-containing surfactants such as Eftop EF301, EF303 (products of Shin-Akita Kasei K.K.), Florade FC430, 431 (products of Sumitomo 3M Co., Ltd.), Megafac F171, F173, F176, F189, R08 (products of Dainippon Ink and Chemicals, Inc.), Surflon S-382, SC101, 102, 103, 104, 105, 106 (products of Asahi Glass Co., Ltd.), and silicon-base surfactants. Also polysiloxane polymer KP-341 (product of Shin-Etsu Chemical Co., Ltd.) and Troysol S-366 (product of Troy Chemical Industries, Inc.) are adoptable as the silicon-base surfactants.

One preferred embodiment of the transfer material prepared according to the method of the invention is a transfer material comprising a temporary support, an alignment layer thereon, and an optically anisotropic layer and a photosensitive polymer layer on the alignment layer. There is no special limitation on materials to be used for preparing the temporary support, and the examples include various polymers such as polyethylene terephthalate. In order to improve detachability of the temporary support or facilitate transferring, a thermoplastic polymer layer, a medium layer or the like may be disposed between the temporary support and the alignment layer.

Next, several embodiments of the optical compensation film, the polarizer plate and the transfer material manufacturable by the method of the present invention will be explained.

[Optical Compensation Film]

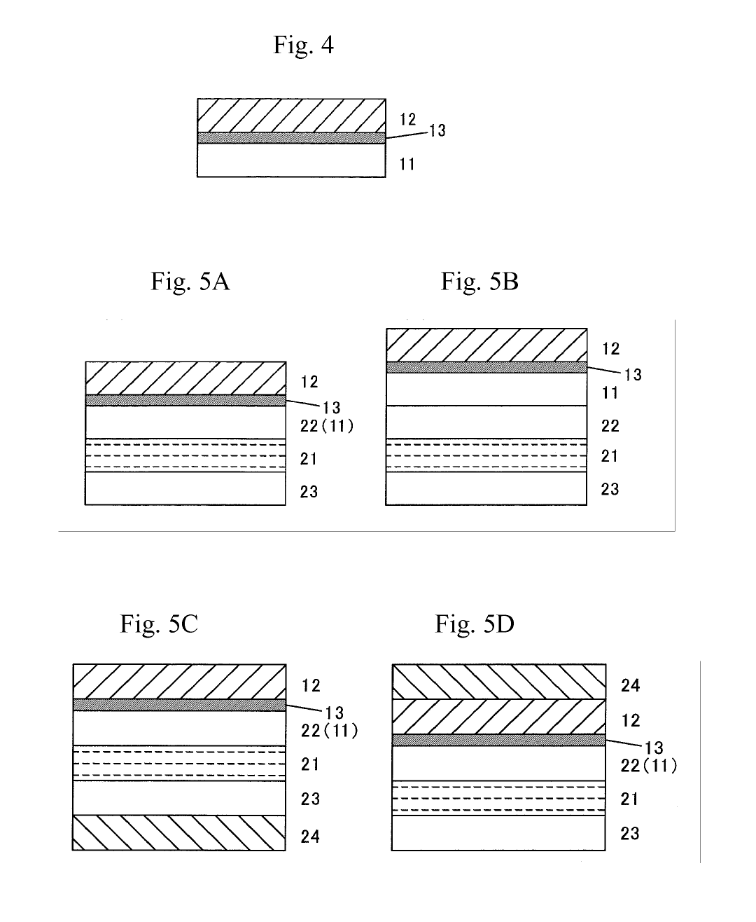

FIG. 4 is a schematic drawing of an exemplary optical compensation film produced by the method of the present invention. The optical compensation film shown in FIG. 4 has an optically anisotropic layer 12 formed on a transparent support 11. Between the transparent support 11 and the cured optically anisotropic layer 12, there is disposed an alignment film 13 used, in the step (2), for controlling alignment of the molecules of the polymerizable liquid crystal compound so as to align them in a first alignment state. Optical characteristics of the optically anisotropic layer 12 may be of so-called biaxial optical anisotropy as described in the above, characterized in that the front retardation (Re) is not zero, and that retardation value measured by making incidence of beam of λ nm in the direction inclined +40° away from the normal line on the optical compensation film while assuming the in-plane slow axis as the axis of inclination (axis of rotation), and retardation value measured by making incidence of beam of λ nm in the direction inclined −40° away from the normal line on the optical compensation film while assuming the in-plane slow axis as the axis of inclination (axis of rotation) are adjusted substantially equal to each other. The optical compensation film of this embodiment is useful to optical compensation of, in particular, VA-mode liquid crystal cell.

The support in the optical compensation film is preferably transparent, and more specifically, preferably composed using a polymer film having a transmittance of 80% or larger. Thickness of the support is preferably 10 to 500 μm, more preferably 20 to 200 μm, and most preferably 35 to 110 μm.

Glass transition temperature (Tg) of the support is appropriately determined depending on purpose of use. The glass transition temperature of the polymer is preferably 70° C. or above, more preferably falls in the range from 75° C. to 200° C., and particularly preferably falls in the range from 80° C. to 180° C. Adoption of any polymer having the glass transition temperature within these ranges is preferable in terms of excellent balance between heat resistance and formability.

Re of the support is preferably adjusted in the range from −200 to 100 nm, and Rth from −100 to 100 nm. Re is more preferably adjusted in the range from −50 to 30 nm, and still more preferably −30 to 20 nm. In this specification, negative Re means that the in-plane slow axis of the support lies in the direction (TD direction) normal to the direction of feeding of the film, and negative Rth means that the thickness-wise refractive index is larger than the in-plane refractive index. In view of improving hue, the in-plane slow axis of the support preferably lies in the TD direction.

Polymers applicable for preparing the support may be, for example, cellulose-base polymers and cycloolefine-base polymers, and more specifically cellulose esters (e.g., cellulose acetate, cellulose propionate, cellulose butyrate), polyolefins (e.g., norbornene-base polymers), poly(meth)acrylic acid ester (e.g., polymethyl methacrylate), polycarbonate, polyester, polysulfone and norbornene-base polymers. Cellulose esters and norbornene-base polymers are preferable in view of low birefringence, wherein the norbornene-base polymers are commercially available under the trade names of Arton (from JSR Corporation), Zeonex, Zeonore (both from Zeon Corporation) and so forth.

For the special case where the support is also used as the protective film of the polarizer film, cellulose esters are preferable, and lower aliphatic acid esters of cellulose are more preferable. The lower aliphatic acids herein mean aliphatic acids having 6 or fewer carbon atoms. The number of carbon atoms is preferably 2 (cellulose acetate), 3 (cellulose propionate) or 4 (cellulose butyrate). It is also possible to use mixed aliphatic acid esters such as cellulose acetate propionate and cellulose acetate butyrate. Among the lower aliphatic acid esters of cellulose, cellulose acetate is most preferable. Degree of substitution of cellulose ester by acyl substituents is preferably 2.50 to 3.00, more preferably 2.75 to 2.95, and most preferably 2.80 to 2.90.

Viscosity-average degree of polymerization (DP) of cellulose ester is preferably 250 or larger, and more preferably 290 or larger. Cellulose ester preferably has a narrow molecular weight distribution expressed by Mm/Mn (Mm is mass-average molecular weight, and Mn is number-average molecular weight) determined by gel permeation chromatography. Value of Mm/Mn preferably falls in the range from 1.0 to 5.0, more preferably from 1.3 to 3.0, and most preferably from 1.4 to 2.0.

Cellulose ester tends to have smaller degrees of substitution at the 6-position, rather than being equally substituted at the 2-, 3- and 6-positions of cellulose. In the present invention, the degree of substitution at the 6-position of cellulose ester is preferably equivalent to, or larger than those at the 2- and 3-positions. Ratio of the degree of substitution at the 6-position to the total degree of substitution at the 2-, 3- and 6-positions is preferably 30 to 40%. Ratio of the degree of substitution at the 6-position is preferably 31% or larger, and particularly preferably 32% or larger. The degree of substitution at the 6-position is preferably 0.88 or larger. The 6-position of cellulose may be substituted by an acyl group having 3 or more carbon atoms (e.g., propionyl, butyryl, valeroyl, benzoyl, acryloyl), besides acetyl group. The degree of substitution at the individual positions can be measured by NMR. Cellulose ester having a high degree of substitution at the 6-position can be synthesized referring to Exemplary Synthesis 1 described in paragraphs 0043 to 0044, Exemplary Synthesis 2 described in paragraphs 0048 to 0049, and Exemplary Synthesis 3 described in paragraphs 0051 to 0052 of Japanese Laid-Open Patent Publication No. H11-5851.

The cellulose ester film may be added with a plasticizer for the purpose of improving mechanical properties, or improving drying speed. Phosphate ester or carboxylate ester may be used as the plasticizer. Examples of the phosphate ester include triphenyl phosphate (TPP), biphenyl diphenyl phosphate and tricresyl phosphate (TCP). Examples of the carboxylate ester include phthalate ester and citrate ester. Examples of the phthalate ester include dimethyl phthalate (DMP), diethyl phthalate (DEP), dibutyl phthalate (DBP), dioctyl phthalate (DOP), diphenyl phthalate (DPP) and diethyl hexyl phthalate (DEHP). Examples of the citrate ester include triethyl O-acetylcitrate (OACTE) and tributyl O-acetylcitrate (OACTB). Examples of other carboxylate ester include butyl oleate, methylacetyl ricinolate, dibutyl sebacate, and various trimellitate esters. Phthalate ester-base plasticizers (DMP, DEP, DBP, DOP, DPP, DEHP) are preferably used. DEP and DPP are particularly preferable. Amount of addition of the plasticizer is preferably adjusted to 0.1 to 25% by mass, more preferably 1 to 20% by mass, and most preferably 3 to 15% by mass, of the content of cellulose ester.

The cellulose ester film may be added with anti-degradation agent (e.g., antioxidant, peroxide decomposing agent, radical inhibitor, metal inactivating agent, acid trapping agent, amine). The anti-degradation agent is described in Japanese Laid-Open Patent Publication Nos. H3-199201, H5-1907073, H5-194789, H5-271471, and H6-107854. Amount of addition of the anti-degradation agent is preferably 0.01 to 1% by mass, more preferably 0.01 to 0.2% by mass, of the solution (dope) to be prepared. Amount of addition less than 0.01% by mass scarcely shows effects of the anti-degradation agent. Amount of addition exceeding 1% by mass may sometimes result in bleeding of the anti-degradation agent onto the surface of film. Particularly preferable examples of the anti-degradation agent can be exemplified by butylated hydroxytoluene (BHT) and tribenzylamine (TBA). It is also possible to add a trace amount of dye for preventing light piping. In consideration of transmittance, species and amount of addition of the dye is preferably adjusted so as to ensure a transmittance of light of 420 nm of 50% or above. Amount of addition of dye is preferably adjusted to 0.01 ppm to 1 ppm.

The cellulose ester film may be added with a retardation control agent for the purpose of controlling Re and Rth. The retardation control agent is preferably used, per 100 parts by mass of cellulose ester, within the range from 0.01 to 20 parts by mass, more preferably from 0.05 to 15 parts by mass, and most preferably from 0.1 to 10 parts by mass. Two or more species of retardation control agents may be used in combination. The retardation control agent is described in the pamphlets of International Patents WO01/88574 and WO00/2619, and in the publications of Japanese Laid-Open Patent Publication Nos. 2000-111914 and 2000-275434.

The cellulose ester film may be produced by the solvent cast process using a solution called “dope”, containing a cellulose ester and other components. The dope may be cast on a drum or a band, and the solvent is then vaporized off to produce the film. Concentration of the dope before being cast is preferably adjusted to have a solid content of 10 to 40% by mass. The solid content is more preferably 18 to 35% by mass. The dope may be cast in two or more layers. The drum or the band is preferably finished to have a specular surface. Methods of casting and drying in the solvent cast process are described in U.S. Pat. Nos. 2,336,310, 2,367,603, 2,492,078, 2,492,977, 2,492,978, 2,607,704, 2,739,069 and 2,739,070, British Patent Nos. 640731 and 736892, Japanese Examined Patent Publication Nos. S45-4554 and S49-5614, and Japanese Laid-Open Patent Publication Nos. S60-176834, S60-203430 and S62-115035.

The dope is preferably cast on the drum or the band having the surface temperature adjusted to 10° C. or below. The cast dope is preferably dried under air flow for 2 seconds or longer. One adoptable method is such as peeling the obtained film off from the drum or the band, and then drying the film under hot air flow at temperatures sequentially varied from 100 to 160° C. so as to vaporize the residual solvent (described in Japanese Examined Patent Publication No. H5-17844). The method can shorten the process time from casting to peeling-off. This method is on the premise that the dope can gelate at the surface temperature of the drum or the band during casting. For the case of casting a plurality of cellulose ester solutions, the film may be produced by casting the cellulose ester-containing solutions respectively from a plurality of casting ports disposed at intervals in the direction of feeding of the support, so as to stack the cast solutions (described in Japanese Laid-Open Patent Publication Nos. S61-158414, H1-122419 and H11-198285). The film may be produced also by casting cellulose ester solutions from two casting ports (described in Japanese Examined Patent Publication No. S60-27562, Japanese Laid-Open Patent Publication Nos. S61-94724, S61-947245, S61-104813, S61-158413 and H6-134933). It is also possible to adopt a method of casting cellulose ester film, by which a high-viscosity and low-viscosity cellulose ester solutions are extruded at the same time, while allowing stream of the low-viscosity cellulose ester solution to surround stream of the high-viscosity cellulose ester solution (described in Japanese Laid-Open Patent Publication No. S56-162617).

The cellulose ester film may be adjusted in the retardation by stretching. The factor of stretching preferably falls in the range from 3 to 100%. Tenter stretching is preferable. In order to precisely control the slow axis, differences in clipping speed and timing of release on both sides of the tenter are preferably minimized as possible. The stretching is described in the pamphlet of International Patent WO01/88574, p. 37, line 8 to p. 38, line 8.

The cellulose ester film may be subjected to surface treatment. The surface treatment can be exemplified by corona discharge treatment, glow discharge treatment, flame treatment, acid treatment, alkali treatment and ultraviolet irradiation treatment. In view of keeping flatness of the film, temperature of the cellulose ester film during the surface treatment is preferably adjusted to Tg (glass transition temperature) or lower, and more specifically 150° C. or below.

For the case where the cellulose ester film is produced by film making process, the thickness thereof is adjustable based on lip flow rate and line speed, or stretching or compression. Because moisture permeability varies depending on the major constituent used therein, control of the film thickness will make the moisture permeability fall in a range suitable for use as the protective film of the polarizer plate. Free volume of the cellulose ester film, produced by film-making process, is adjustable by temperature and time of drying. Because moisture permeability again varies depending on the major constituent used therein, control of the free volume will make the permeability fall in a range suitable for use as the protective film. Hydrophilicity and hydrophobicity of the cellulose ester film are adjustable by additives. Addition of any hydrophilic additive into the free volume increases the moisture permeability, and conversely addition of any hydrophobic additive decreases the moisture permeability. As described in the above, the moisture permeability of the cellulose ester film is adjustable by various methods to a desirable range suitable for use as the protective film of the polarizer plate, and thereby the support of the optically anisotropic layer can now serve also as the protective film of the polarizer plate, making it possible to manufacture the polarizer plate having an optical compensation function at low costs and high productivity.

[Polarizer Plate]

FIGS. 5A to 5D are schematic sectional views of the polarizer plate having the optical film produced by the method of the present invention. The polarizer plate is produced generally by dying a polarizer film composed of a polyvinyl alcohol film with iodine, stretching the film to obtain a polarizer film 21, and stacking protective films 22 and 23 on both sides thereof. If an optical film having a support having an optically anisotropic layer supported thereon, typically composed of a polymer film, is used, the support can directly be used as at least one of the protective films 22 and 23. The optically anisotropic layer 12 in this case may be disposed on the polarizer layer 21 side (that is, the optically anisotropic layer 12 is more closer to the polarizer layer 21 rather than to the support 11), or may be disposed on the opposite side of the polarizer layer 21 (that is, the optically anisotropic layer 12 is more distant from the polarizer layer 21 rather than from the support 11), wherein the optically anisotropic layer 12 is preferably on the opposite side of the polarizer layer 21, as shown in FIG. 5A. Alternatively, as shown in FIG. 5B, the optically anisotropic layer 12 may be bonded to the external of one protective film 22 on the polarizer layer 21, typically using a pressure-sensitive adhesive in between.

FIGS. 5C and 5D show exemplary configurations of a polarizer plate configured as shown in FIG. 5A, further having other functional layers 24 disposed thereon. FIG. 5C shows an exemplary configuration having other functional layer 24 disposed on the protective film 23 disposed on the opposite side of the optical compensation film of the present invention, while placing the polarizer layer 21 in between, and FIG. 5D is an exemplary configuration having other functional layer 24 disposed on the optical compensation film of the present invention. The other functional layer is not specifically limited, and is exemplified by functional layers capable of imparting various characteristics, such as quarter-wave layer, anti-reflection layer and hard-coat layer. These layers may be bonded as one component of quarter-wave plate, anti-reflection film and hard-coat film typically using a pressure-sensitive adhesive, or as shown by an exemplary configuration in FIG. 5D, may be formed preliminarily on the optical compensation film (optically anisotropic layer 12) of the present invention, and then bonded to the polarizer layer 21. It is also possible that the protective film 23 as itself, on the opposite side of the optical compensation film of the present invention, may be configured as the other functional film such as quarter-wave plate, anti-reflection film and hard-coat film.

The polarizer film can be exemplified by iodine-containing polarizer film, dye-containing polarizer film using dichroic dye, and polyene-base polarizer film. The iodine-containing polarizer film and the dye-containing polarizer film are produced generally by using a polyvinyl alcohol-base film. Species of the protective film is not specifically limited, allowing use of cellulose esters such as cellulose acetate, cellulose acetate butyrate and cellulose propionate; polycarbonate; polyolefin; polystyrene; polyester and so forth. A transparent protective film is supplied generally in a form of roll, and is preferably bonded in a continuous manner while making the longitudinal direction (MD) thereof coincide with the web-form polarizer film. Axis of alignment (slow axis) of the protective film herein may have any direction. Also angle between the slow axis (axis of alignment) of the protective film and the absorption axis (axis of stretching) of the polarizer film is not specifically limited, and may appropriately be set depending on purposes of the polarizer plate.

The polarizer film and the protective film may be bonded using a water-base adhesive. Solvent contained in the water-base adhesive is dried in the process of diffusion through the protective film. The larger the permeability of the protective film will be, the faster the drying will be and the higher the productivity will be, but too large permeability will degrade the polarization performance if moisture enters the polarizer film due to (highly humid) environment of use of the liquid crystal display device. The moisture permeability of the optical compensation film is determined typically by the thickness, free volume, and hydrophilicity/hydrophobicity of the polymer film (and polymerizable liquid crystal compound). The moisture permeability of the protective film of the polarizer plate preferably falls in the range from 100 to 1000 (g/m²)/24 hrs, and more preferably from 300 to 700 (g/m²)/24 hrs.

In the present invention, one of the protective films of the polarizer film may be used also as a support of the optically anisotropic layer, for the purpose of thinning or the like. The optical compensation film and the polarizer film are preferably fixed, in view of avoiding shifting of the optical axes, and of preventing dust or other foreign matters from entering. Appropriate methods such as placing a transparent adhesive layer in between may be adoptable for the fixation and stacking. Species of the adhesive or the like are not specifically limited, wherein those in no need of high temperature processes for curing and drying in the adhesion process are preferable, and also those in no need of long duration of time for the curing and drying are preferable. From this point of view, adhesives and pressure-sensitive adhesives of hydrophilic polymer base are preferably used.

It is also possible to use the polarizer plate having an appropriate functional layer, formed on one surface or on both surfaces of the polarizer film, such as a protective film aimed at various purposes including water-proofness equivalent to that of the above-described protective film, an anti-reflection layer aimed at preventing surface reflection and/or an anti-glare layer. The anti-reflection layer can appropriately be formed typically as a light interferential film composed of a coated layer of a fluorine-containing polymer, or a multi-layered metal evaporated film. The anti-glare layer can be formed according to an appropriate system capable of diffusing surface reflective light, by providing a micro-irregularity structure to the surface typically by forming a coated layer of particle-containing polymer, embossing, sand blasting, etching or the like.

The particle appropriately adoptable herein is any one species or two or more species selected from inorganic particles of silica, calcium oxide, alumina, titania, zirconia, tin oxide, indium oxide, cadmium oxide, antimony oxide, and the like, having a mean particle size of 0.5 to 20 μm, being occasionally electro-conductive, and crosslinked or un-crosslinked organic particles composed of appropriate polymers such as polymethyl methacrylate and polyurethane. The adhesive layer and the pressure-sensitive adhesive layer may be such as those showing light diffusing property by virtue of such particle contained therein.

The polarizer plate of the present invention preferably has optical properties and durability (short-term and long-term storability) equivalent to, or better than those of a commercially-available, super-high-contrast product (for example, HLC2-5618 from Sanritz Corporation). More specifically, the polarizer plate preferably has a visible light transmissivity of 42.5% or above, a degree of polarization of {(Tp−Tc)/(Tp+Tc)}^{1/2}≧0.9995 (where, Tp represents parallel transmissivity, and Tc represents orthogonal transmissivity), and the rate of change in the transmissivity before and after the polarizer plate was allowed to stand in an atmosphere of 60° C., 90% RH for 500 hours, and then in a dry atmosphere of 80° C. for 500 hours, is 3% or below on the basis of the absolute value, and more preferably 1% or below, whereas the rate of change in the degree of polarization is 1% or below on the basis of the absolute value, and more preferably 0.1% or below.

[Transfer Material]

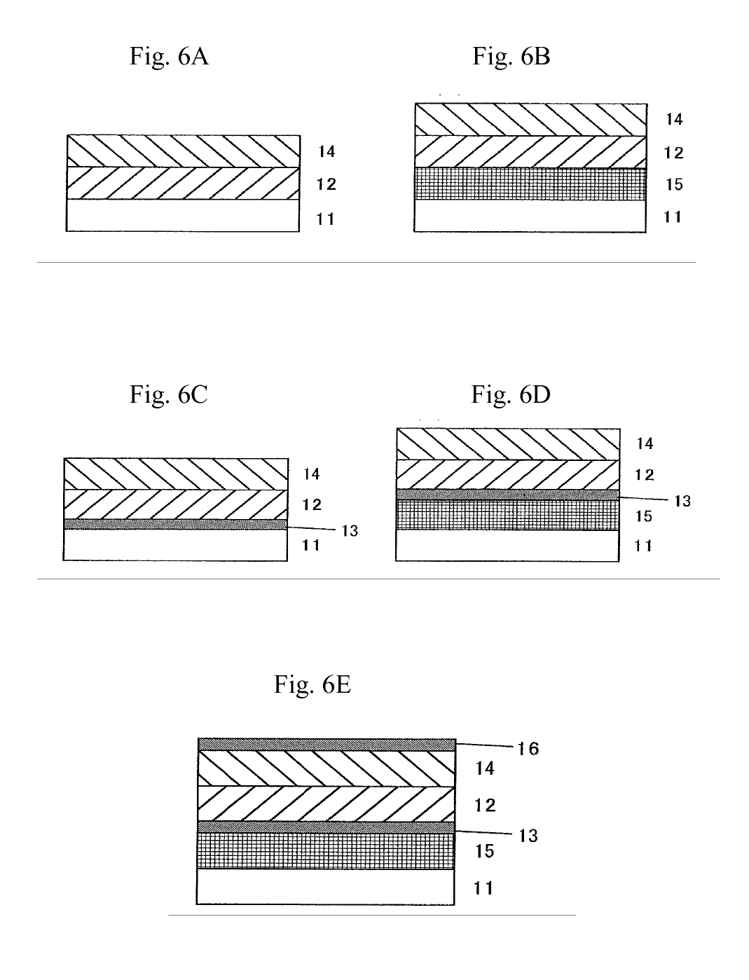

FIGS. 6A to 6E are schematic sectional views of the transfer material of the present invention, produced by forming a photosensitive polymer layer on the optical film produced by the method of the present invention. The transfer material of the present invention has a support, at least one optically anisotropic layer, and at least one photosensitive polymer layer, aimed at being used for transferring at least the optically anisotropic layer and the photosensitive polymer layer onto another substrate. The transfer material of the present invention shown in FIG. 6A has an optically anisotropic layer 12 and a photosensitive polymer layer 14 formed on a transparent or opaque temporary support 11. The transfer material of the present invention may have any other layers, and may have, for example as shown in FIG. 6B, a layer 15 aimed at controlling mechanical characteristics or at imparting conformance to surface irregularity, such as cushioning for absorbing irregularity on the opposing substrate side in the transfer process, between the temporary support 11 and the optically anisotropic layer 12, or may have, as shown in FIG. 6C, a layer 13 functioning as an alignment layer controlling alignment of liquid crystal molecules in the optically anisotropic layer 12, or still may have, as shown in FIG. 6D, both of these layers. It is still also possible to provide, as shown in FIG. 6E, a separable protective layer 16 on the topmost surface, for the purpose of surface protection of the photosensitive polymer layer.

[Target Substrate for Transfer for Composing Liquid Crystal Display Device]

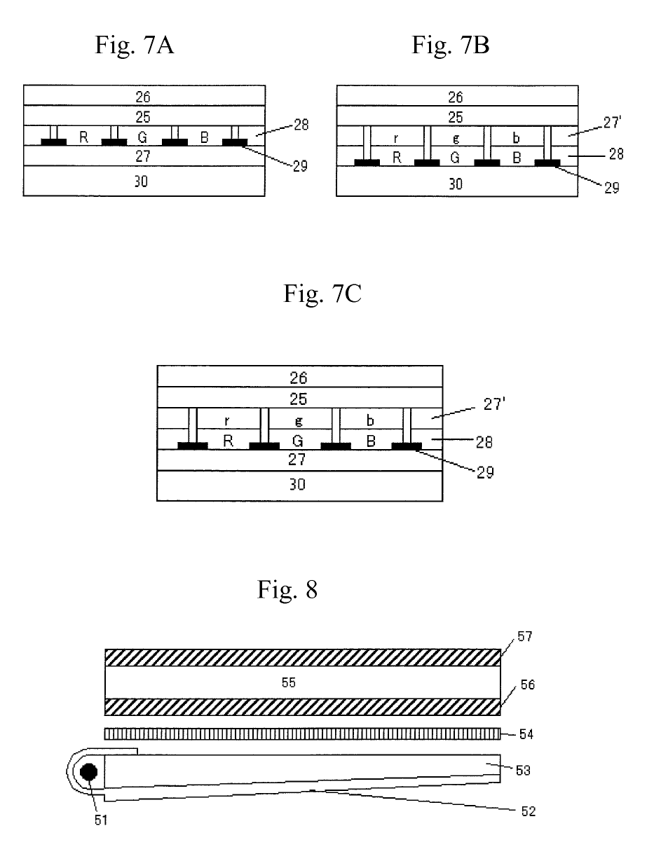

The transfer material of the present invention is transferred onto substrates composing liquid crystal display device, and can configure the optically anisotropic layer contributive to compensation of viewing angle of the liquid crystal cell. The transfer material combined with color filters can also configure the optically anisotropic layer contributive to color-wise compensation, for R, G and B, of viewing angle of the liquid crystal cell. The substrate having these layers transferred thereon may be used for any one of, or both of a pair of substrates composing the liquid crystal cell. FIG. 7A is a schematic sectional view showing an exemplary substrate having the optically anisotropic layer transferred thereon, produced using the transfer material of the present invention. A target substrate 30 for transfer is not specifically limited so far as it is transparent, wherein birefringence thereof is preferably small, and is therefore composed using glass or low-birefringent polymer. On the substrate, there is an optically anisotropic layer 27 formed by using the transfer material of the present invention, and further thereon a black matrix 29, and color filter layers 28 are formed. Although not shown in FIG. 7A, a photosensitive polymer layer which is a constituent layer of the transfer material is disposed between the optically anisotropic layer 27 and the substrate 30, wherein the optically anisotropic layer 27 and the substrate 30 are bonded while placing the photosensitive polymer layer in between. A transparent electrode layer 25 is formed further on the color filter layers 28, and an alignment layer 26 aligning the liquid crystal molecules in the liquid crystal cell is formed still further thereon. The black matrix 22 and the color filter layers 28 may be formed, after the optically anisotropic layer 27 is formed on the substrate 30 using the transfer material of the present invention, by uniformly coating a resist, irradiating the resist with light through a mask, and developing the resist to thereby remove unnecessary portion, or may be formed by printing system or ink-jet system proposed recently. The latter is preferable in view of cost.

FIG. 7B is a schematic sectional view showing an exemplary substrate having the color filter combined with the optically anisotropic layer, produced by using the transfer material of the present invention. The target substrate 30 for transfer is not specifically limited so far as it is transparent, wherein birefringence thereof is preferably small, and is therefore composed using glass or low-birefringent polymer. The substrate generally has the black matrix 29 formed thereon, and further thereon the color filter layers 28 and the optically anisotropic layer 27′ composed of the photosensitive polymer layer which was transferred from the transfer material of the present invention, and was patterned typically by light exposure through a mask, are formed. FIG. 4 shows an embodiment having color filter layers 28 for R, G and B formed therein, whereas as being often found recently, the color filter layers composed of layers for R, G, B and W (white) may be formed. The optically anisotropic layer 27′ is divided into r, g and b regions, being optimized in the retardation property with respect to each of R, G and B colors of the individual filter layers 28. Any other layer transferred from the transfer material may reside on the optically anisotropic layer 27′, but it is preferably removed in the process of development and rinsing, in view of avoiding as possible contamination of the liquid crystal cell with impurities. The transparent electrode layer 25 is formed on the optically anisotropic layer 27′, and further thereon, the alignment layer 26 aligning the liquid crystal molecules in the liquid crystal cell is formed.

It is still also possible, as shown in FIG. 7C, to form both of the unpatterned solid optically anisotropic layer 27 and the patterned optically anisotropic layer 27′ on a single substrate, using the transfer material of the present invention. Although not shown in the drawing, a solid optically anisotropic layer 27 may be formed on one of a pair of opposed substrates of the liquid crystal cell, and a patterned optically anisotropic layer 27′ may be formed together with the color filter layers 28 on the other substrate, using the transfer material of the present invention. One of the pair of opposed substrates often has, in general, a drive electrode typically composed of a TFT array, so that the solid optically anisotropic layer 27 may be formed on the drive electrode, or the patterned optically anisotropic layer 27′ may be formed together with the color filter layers 28 on the drive electrode. Although formation at any levels on the substrate is possible, the optically anisotropic layer in the active-matrix-type device is preferably formed on the upper side of the silicon layer, considering heat resistance of the optically anisotropic layer.

By using the transfer material of the present invention, a single cycle of transfer-exposure-development process can form a filter of a single color and corresponding optically anisotropic layer at the same time, so that the viewing angle dependence of the liquid crystal display device can be improved by the same number of process steps as that in the process of producing a color filter described in Japanese Laid-Open Patent Publication No. H3-282404.

[Liquid Crystal Display Device]

FIG. 8 shows an exemplary liquid crystal display device adopting the polarizer plate of the present invention. The liquid crystal display device has a liquid crystal cell 55 having nematic liquid crystal held between the upper and lower electrode substrates, and a pair of polarizer plates 56 and 57 disposed on both sides of the liquid crystal cell, wherein at least one of the polarizer plates is configured using the polarizer plate of the present invention shown in FIGS. 5A to 5D. The polarizer plate of the present invention may be disposed so as to locate the optically anisotropic layer between the polarizer layer and the electrode substrate of the liquid crystal cell. The nematic liquid crystal molecules are controlled as keeping a predetermined state of alignment, with the aid of the alignment film provided on the electrode substrate and rubbed on the surface thereof, or with the aid of provision of a structure such as rib.

The device may have one or more light conditioning films 54 such as luminance improving film and diffuser film, on the lower side of the liquid crystal cell held between the polarizer plates. On the further lower side of the light conditioning film, the device has a reflective plate 52 reflecting light emitted from a cold cathode ray tube 51 back to the front, and a light guide plate 53. In place of a back light unit composed of the cold cathode ray tube and the light guide plate, recent trends relate to a direct back light having a plurality of cold cathode ray tubes arrayed under the liquid crystal cell, an LED back light using LED as a light source, and a back light based on surface emission making use of organic EL, inorganic EL or the like, all of which being adoptable to the present invention.

Although not shown in the drawing, only a single polarizer plate will sufficiently be disposed on the observer's side in the embodiment of the reflection-type liquid crystal display device, wherein the reflective film is disposed on the back surface of the liquid crystal cell or on the inner surface of the lower substrate of the liquid crystal cell. Of course, a front light using the above-described light source may be provided on the observer's side of the liquid crystal cell. It is still also possible to configure the display device as of semi-transparent type, providing both of a transmissive portion and a reflective portion to a single pixel of the display device.

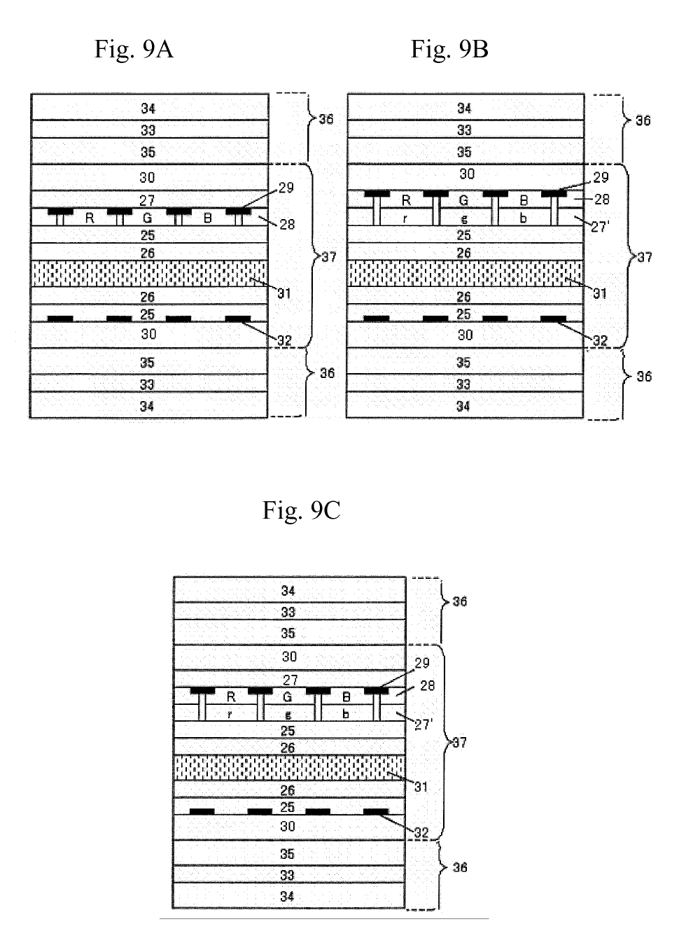

FIGS. 9A to 9C are schematic sectional views of an exemplary liquid crystal display devices using the transfer material of the present invention. The liquid crystal display devices shown in FIG. 9A to 9C are those configured by using liquid crystal cells 37, composed of the glass substrates shown in FIG. 7A to 7C, respectively, as the upper substrate, a glass substrate having a TFT layer 32 formed thereon as an opposing substrate, and a liquid crystal 31 held therebetween. On both sides of the liquid crystal cell 37, polarizer plates 36, each composed of a polarizer layer 33 and two cellulose ester films 34, 35 holding it in between, are disposed. The cellulose ester film 35 on the liquid crystal cell side may be configured by using an optical film contributive to optical compensation, or may have only a function of a protective film, similarly to 34. Although not shown in the drawing, only one polarizer plate will sufficiently be disposed on the observer's side in the embodiment of the reflection-type liquid crystal display device, wherein the reflection film is disposed on the back surface of the liquid crystal cell, or on the inner surface of the lower substrate of the liquid crystal cell. Of course, a front light may be provided on the observer's side of the liquid crystal cell. It is still also possible to configure the display device as of semi-transparent type, providing both of a transmissive portion and a reflective portion to a single pixel of the display device. Display mode of the liquid crystal display device is not specifically limited, and the present invention is applicable to all transmission-type and reflection-type liquid crystal display devices, such as VA-mode, STN-mode, TN-mode and OCB-mode liquid crystal display device. Among others, the present invention is effective when applied to the VA-mode device for which improvement in viewing angle dependence of color is strongly desired.

The VA-mode liquid crystal cell is configured as having liquid crystal molecules of negative dielectric anisotropy confined between the upper and lower substrates rubbed on the opposing surfaces thereof. For example, by using liquid crystal molecules having Δn=0.0813 and Δ∈=−4.6 or around, a liquid crystal cell having a director, indicating direction of alignment of the liquid crystal molecules, or so-called tilting angle, of approximately 89° may be produced. In this case, the thickness d of the liquid crystal layer may be adjusted to 3.5 μm or around. Brightness in the white state varies depending on product Δn·d of the thickness d (nm) of the liquid crystal layer, and the refractive index anisotropy Δn. In order to obtain the maximum brightness, the thickness d of the liquid crystal layer is preferably adjusted to the range from 2 to 5 μm (2000 to 5000 nm), and Δn is preferably adjusted to the range from 0.060 to 0.085.

The upper and lower substrates of the liquid crystal cell have transparent electrodes formed on the inner surfaces thereof, wherein in the non-driving state having no drive voltage applied to the electrodes, the liquid crystal molecules in the liquid crystal layer align nearly normal to the surfaces of the substrates, so that the state of polarization of light passing through the liquid crystal panel hardly changes. Because the absorption axis of the upper polarizer plate of the liquid crystal crosses nearly normal to the absorption axis of the lower polarizer plate, light does not pass through the polarizer plates. In this way, the VA-mode liquid crystal display device can realize an ideal black state in the non-driving state. On the contrary in the driving state, the liquid crystal molecules incline in the direction parallel with the surface of the substrates, so that light passing through the liquid crystal panel is modified in the state of polarization thereof by the inclined liquid crystal molecules, and can pass through the polarizer plates.

The foregoing paragraphs has been discussing the case where the electric field is applied between the upper and lower substrates, and therefore a liquid crystal material having a negative dielectric anisotropy, molecules of which being capable of responding normal to the direction of electric field, was used. It is, however, also possible to use a liquid crystal material having a positive dielectric anisotropy, for the case where the electric field is applied in the transverse direction, which is in parallel with the surface of the substrates.

Advantages of the VA mode are rapid response and high contrast. The contrast high in the front view, however, degrades in oblique views. The liquid crystal molecules in the black state align normal to the surface of the substrates, and show almost no birefringence in the front view, so that low transmissivity and high contrast can be obtained. However in oblique views, the liquid crystal molecules raise birefringence. The angle of crossing of the absorption axes of the upper and lower polarizer plates, orthogonally 90° in the front view, becomes larger than 90° in oblique views. Due to two these reasons, the VA mode is more likely to cause leakage light, degrading the contrast. The present invention can solve these problems by disposing the optical compensation film of the present invention between the liquid crystal cell and the polarizer plates, by using the polarizer plate of the present invention, and/or by incorporating (preferably into the liquid crystal cell) at least one optically anisotropic layer transferred from the transfer material of the present invention.

The VA mode, having the liquid crystal molecules thereof inclined in the white state, causes difference in the luminance and hue between the view in the direction of inclination and the view in the opposite direction, because the liquid crystal molecules show different degrees of birefringence in the oblique views. To solve this problem, the liquid crystal cell is preferably configured as adopting a multi-domain system. The multi-domain system relates to a structure having a plurality of regions, differed in the state of alignment, formed in a single pixel. For example, in the VA-mode liquid crystal cell based on the multi-domain system, a plurality of regions, differed from each other in the state of angle of inclination of the liquid crystal molecules under application of electric field, reside in a single pixel. The VA-mode liquid crystal cell based on the multi-domain system can average the angle of inclination of the liquid crystal molecules under application of electric field in a pixel-by-pixel manner, and can thereby average the viewing angle dependence. Separation of alignment within a single pixel can be accomplished by providing slits to the electrodes, by providing projections, by altering the direction of electric field, or by biasing the density of electric field. Although larger numbers of division may contribute to more uniform viewing angle dependence in all directions, quadrisection is preferable in view of avoiding lowering in the transmissivity in the white state.

A chiral agent generally used for the twisted nematic (TN)-mode liquid crystal display devices is used not so often for the VA-mode liquid crystal display devices because of possible degradation in the dynamic response characteristics, but may be added in order to reduce alignment failure. The liquid crystal molecules are hard to response in the boundary regions of division of alignment. This may lower the luminance in the normally-black display having the black state maintained therein. Addition of the chiral agent to the liquid crystal material may contributes to reduce the boundary regions.

### EXAMPLES

The present invention will further specifically be explained referring to Examples. Any materials, reagents, amounts and ratios of substances, operations and so forth may appropriately be modified without departing from the spirit of the present invention. It is therefore to be understood that the present invention is by no means limited to the specific examples below.

### Examples 1 to 5, and Comparative Example 1

### Manufacture of Optical Film

(Manufacture of Transparent Support S-1)

Fujitac TD80UF (from Fujifilm Corporation, Re=3 nm, Rth=50 nm), which is a commercially-available cellulose acetate film, was used as a transparent support S-1.

(Preparation of Coating Liquid AL-1 for Forming Alignment Layer)

The composition below was prepared, filtered through a polypropylene filter having a pore size of 30 μm, and the resultant filtrate was used as a coating liquid AL-1 for forming the alignment layer. Modified polyvinyl alcohol used herein was such as described in Japanese Laid-Open Patent Publication No. H9-152509.

| Formulation of Coating Liquid for | |
|---|---|
| Forming Alignment Layer | (% by mass) |
| Modified polyvinyl alcohol AL-1-1 | 4.01 |
| Water | 72.89 |
| Methanol | 22.83 |
| Glutaraldehyde (crosslinking agent) | 0.20 |
| Citric acid | 0.008 |
| Monoethyl citrate | 0.029 |
| Diethyl citrate | 0.027 |
| Triethyl citrate | 0.006 |

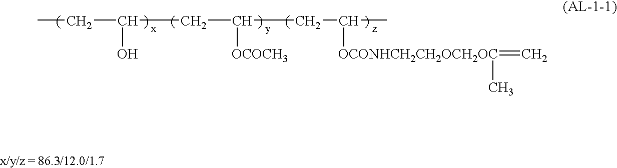 (Preparation of Coating Liquid AL-2 for Forming Intermediate Layer/Alignment Layer)

The composition below was prepared, filtered through a polypropylene filter having a pore size of 30 μm, and the resultant filtrate was used as a coating liquid AL-2 for forming the intermediate layer/alignment layer for separation.

| Formulation of Coating Liquid | |
|---|---|
| for Forming Intermediate Layer/Alignment Layer | (% by mass) |
| Polyvinyl alcohol (PVA205, from Kuraray Co., Ltd.) | 3.21 |
| Polyvinyl pyrrolidone (Luvitec K30, FROM BASF) | 1.48 |
| Distilled water | 52.1 |
| Methanol | 43.21 |

(Preparation of Coating Liquid LC-1 for Forming Optically Anisotropic Layer)

The composition shown in the table below was prepared, filtered through a polypropylene filter having a pore size of 0.2 μm, and the resultant filtrate was used as a coating liquid LC-1 for forming the optically anisotropic layer. In the table, LC242 is a rod-like liquid crystal (polymerizable liquid crystal, Paliocolor LC242, from BASF Japan), and LC756 is a chiral agent (Paliocolor LC756, from BASF Japan). Dichroic photo-polymerization initiator LC-1-1 was synthesized according to the method described in EP1388538A1, page 21. Horizontal alignment agent LC-1-2 was synthesized referring to the method described in Tetrahedron Lett., Vol. 43, p. 6793 (2002).

| Formulation of Coating Liquid | |
|---|---|
| for Forming Optically Anisotropic Layer | (% by mass) |
| Rod-like liquid crystal (Paliocolor LC242, from | 33.37 |
| BASF Japan) | |
| Chiral agent (Paliocolor LC756, from BASF Japan) | 3.10 |
| Photopolymerization initiator (LC-1-1) | 1.55 |
| LC-1-2 | 0.08 |
| Diazoxy dianisole | 0.50 |
| Methyl ethyl ketone | 61.40 |

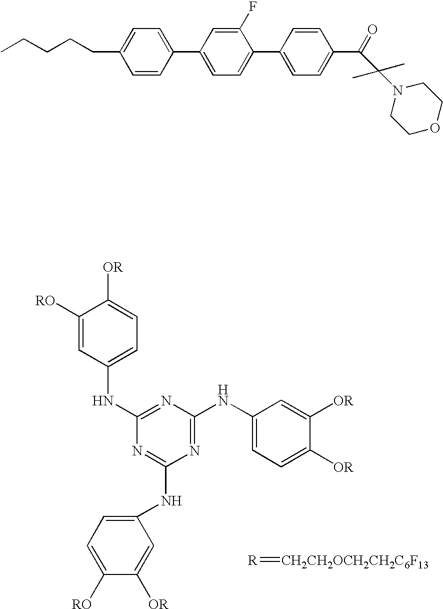 (Preparation of Coating Liquid CU-1 for Forming Thermoplastic Polymer Layer)

The composition shown in the table below was prepared, filtered through a polypropylene filter having a pore size of 30 μm, and the resultant filtrate was used as a coating liquid CU-1 for forming the thermoplastic polymer layer.

| Formulation of Coating Liquid | |
|---|---|
| for Forming Thermoplastic Polymer Layer | (% by mass) |
| Methyl methacrylate/2-ethylhexyl acrylate/benzyl | 5.89 |
| methacrylate/methacrylic acid copolymer (compositional | |
| ratio of copolymerization (molar ratio) = 55/30/10/5, | |
| weight average molecular weight = 100,000, Tg≈70° C.) | |
| Styrene/acrylic acid copolymer (compositional ratio | 13.74 |
| of copolymerization (molar ratio) = 65/35, weight average | |
| molecular weight = 10,000, Tg≈100° C.) | |
| BPE-500 (from Shin-Nakamura Chemical Co., Ltd.) | 9.20 |
| Megafac F-780-F (from Dainippon Ink and Chemicals, Inc.) | 0.55 |
| Methanol | 11.22 |
| Propylene glycol monomethyl ether acetate | 6.43 |
| Methyl ethyl ketone | 52.97 |

(Preparation of Coating Liquid PP-1 for Forming Photosensitive Polymer Layer)

The composition shown in the table below was prepared, filtered through a polypropylene filter having a pore size of 0.2 μm, and the resultant filtrate was used as a coating liquid PP-1 for forming the photosensitive polymer layer.

| Formulation of Coating Liquid | |
|---|---|
| for Forming Photosensitive Polymer Layer | (% by mass) |
| Random copolymer having a molar ratio of benzyl | 5.0 |
| methacrylate/methacrylic acid = 72/28, by molar ratio | |
| (weight-average molecular weight = 37,000) | |
| Random copolymer of benzyl methacrylate/methacrylic | 2.45 |
| acid = 78/22, by molar ratio (weight-average | |
| molecular weight = 40,000) | |
| KAYARAD DPHA (from Nippon Kayaku Co., Ltd.) | 3.2 |
| Radical polymerization initiator (Irgacure 907, from Ciba | 0.75 |
| Specialty Chemicals) | |
| Sensitizer (Kayacure DETX, from Nippon Kayaku Co., | 0.25 |
| Ltd.) | |
| Cationic polymerization initiator (diphenyl iodonium | 0.1 |
| hexafluorophosphate, from Tokyo Chemical Industry | |
| Co., Ltd.) | |
| Propylene glycol monomethyl ether acetate | 27.0 |
| Methyl ethyl ketone | 53.0 |
| Cyclohexanone | 9.1 |
| Megafac F-176PF (from Dainippon Ink and Chemicals, | 0.05 |
| Inc.) | |

(One-Side Saponification of Cellulose Ester Film)

A cellulose ester film was allowed to pass through induction heating rolls at 60° C. so as to elevate the surface temperature of the film to 40° C., then 14 ml/m² of an alkali solution having the composition shown below was coated using a bar coater. The film was allowed to stand for 10 seconds under a steam-type far infrared heater (from Noritake Co., Ltd.) heated to 110° C., and thereon 3 ml/m² of pure water was coated using the same bar coater. Film temperature in this process was 40° C. Cleaning with water using a fountain coater and dewatering using an air knife were then repeated three times, and the film was allowed to stand for two seconds in a 70° C. drying zone for drying.

(Polarized UV Irradiation Apparatuses POLUV-1, POLUV-2)

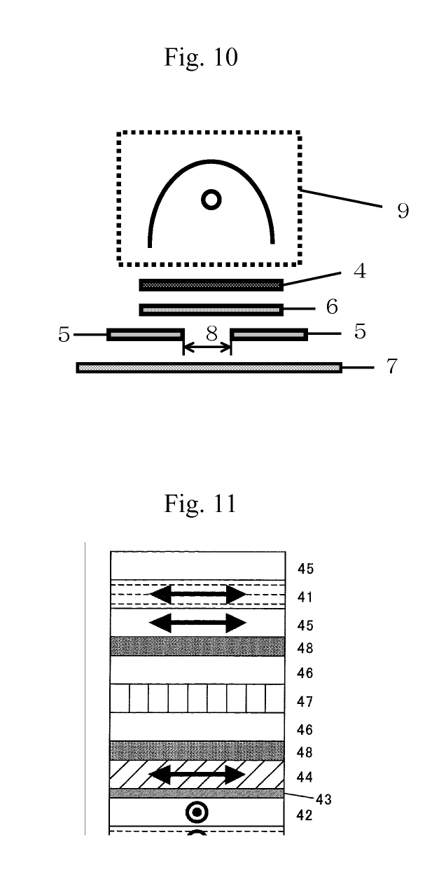

As shown in FIG. 10, a polarized UV irradiation apparatus POLUV-1 was produced by using a ultraviolet irradiation apparatus (Light Hammer 10, 240 W/cm, from Fusion UV Systems) 9, based on the microwave emission system equipped with a D-bulb having an intense emission spectrum at 350 to 400 nm, as a light source unit, by disposing a wavelength selection filter 4 (short wavelength cut filter LU0350, from Asahi Spectra Co., Ltd.) 4 cm away from the irradiation surface, by disposing a wire-grid polarizer filter 6 (ProFlux PPL02 (high transmissivity type), from Moxtek Inc.) 3 cm away from the irradiation surface, and by disposing an aperture 5, composed of two 50 mm×50 mm aluminum plates, 2.5 cm away from the irradiation surface. A polarized UV irradiation apparatus POLUV-2 was also produced by replacing the wire-grid polarizer filter in POLUV-1 with a dielectric mirror (ultra-wide-range dielectric planar mirror TFMS-50C8-4/11, from Sigma Koki Co., Ltd.).

(Measurement of Energy Density and Intensity)

Irradiation corresponded to Examples 1, 2, and Comparative Examples 1 to 4 using polarized ultraviolet light was carried out under conditions listed in Table 1. For the measurement of intensity and extinction ratio, an intensity meter (UVPF-A1, from Eyegraphics Co., Ltd.), and a wire-grid polarizer filter (ProFlux PPL02 (high transmittance type), from Moxtek, Inc.), as the polarizer and analyzer, were used.

One surface of the transparent support S-1 was saponified as described in the above, thereon the coating liquid AL-1 for forming the alignment layer was coated using a #14 wire bar coater, dried under a hot air of 60° C. for 60 seconds, further dried under a hot air of 90° C. for 150 seconds, to thereby form an alignment layer of 1.0 μm thick. Next, thus-formed alignment layer was rubbed in the moving direction (MD) of the transparent support, thereon the coating liquid LC-1 for forming the optically anisotropic layer was coated using a #7 wire bar coater, then dried and ripened under heating at a temperature of film surface of 95° C. (measured using an infrared radiation thermometer IT2-01, from Keyence Corporation, the same will apply hereinafter) for 2 minutes, to thereby form an optically anisotropic layer having a uniform liquid crystal phase. Immediately after the ripening, polarized ultraviolet light was irradiated on the optically anisotropic layer while keeping a temperature of film surface of 80° C., under a nitrogen atmosphere with an oxygen concentration of 0.3%, using the polarized UV irradiation apparatuses POLUV-1 and POLUV-2, according to the conditions listed in Table 1, to thereby manufacture optical films of Examples 1, 2 and Comparative Examples 1 to 4. The feeding speed of the support in the process of irradiation of polarized ultraviolet light was adjusted to 5 m/min. The optically anisotropic layer after being fixed showed no liquid crystallinity even under elevated temperatures.

Isotropic phase transition temperature of the rod-like liquid crystal (Paliocolor LC242, from BASF Japan) used in LC-1 was found to be 100.2° C.

TABLE 1

| | | | Percentage of | Energy density |
|---|---|---|---|---|
| | | | components of | under a |
| | Types of | Slit width | light with | condition of |
| | polarized | (8 in | extinction ratio | feeding a support |
| | ultraviolet | FIG. 1) | of 1 to 8 | at 5 m/min. |
| | radiation | mm | % | mJ/cm² |
| Example 1 | POLUV-1 | 60 | 13 | 322 |
| Example 2 | POLUV-1 | 40 | 7 | 276 |
| Example 3 | POLUV-1 | 20 | 5 | 173 |
| Example 4 | POLUV-1 | 10 | 5 | 91 |
| Example 5 | POLUV-2 | 40 | 5 | 50 |
| Comparative | POLUV-1 | 90 | 17 | 345 |
| Example 1 | | | | |

As shown in Table 1, the samples were successfully prevented from being irradiated by components of light having small extinction ratios (ranging from 1 to 8), by adjusting the slit width, proving feasibility of the method of the present invention. As described in the above, it is understandable from the results shown in Table 1 that narrowing of the slit width resulted in lowered intensity and reduced the light energy density given on the samples, but energy of irradiation capable of forming the optically anisotropic layer with sufficient strength can be obtained by adjusting the feeding speed and so forth.

It is also understandable from Table 1 that the dielectric mirror polarizer used in Comparative Example 2 allows only a smaller energy density as compared with the wire-grid polarizer, due to poor availability of light, as described in Published Japanese Translation of PCT International Publication for Patent Application No. 2002-512850.

(Measurement of Retardation)

Front retardation Re of the samples, and retardations Re(40), Re(−40) of the samples inclined by ±40′ assuming the slow axes thereof as the axis of rotation were measured at 589 nm, using KOBRA 21ADH (from Oji Scientific Instruments). Retardation of the optically anisotropic layer was determined by subtracting retardation of the support at each angle from retardation of the optical compensation film as a whole at each angle.

(Anti-Scratching Test)

Scratching test was carried out using a rubbing tester, according to the conditions below:

Environmental conditions for evaluation: 25° C., 60% RH;

Rubbing material: Dusper (Ozu Paper Co., Ltd.) was wound round the rubbing tip (1 cm×1 cm) of the tester to contact with the samples, and immobilized with a band;

Moving distance (one-way): 10 cm;

Rubbing speed: 13 cm/second;

Load: 500 g/cm²;

Contact area of tip: 1 cm×1 cm; and

Number of times of rubbing: 50 round trips.

An oil-base black ink was painted on the back surface of thus-rubbed samples, and scratches on the rubbed portion was visually observed by reflected light, and evaluated according to the criteria below:

⊚: no scratches seen at all even if observed with the greatest care;

∘: shallow scratches slightly seen when observed with the greatest care;

Δ: shallow scratches slightly seen; and

x: scratches seen.

(Evaluation of Surface Condition)

The optical film, held between the polarizer plates in the cross-nicol configuration, was placed on a schaukasten, and alignment of the liquid crystal was confirmed.

∘: good alignment over the entire surface;

Δ: alignment partially disturbed; and

x: alignment disturbed over the entire surface.

### Comparison among Examples 1 and 2, and Comparative Example 1

Results of measurement of retardation, surface condition, and anti-scratching test are shown in Table 2.

TABLE 2

| | | | | | Scratching |
|---|---|---|---|---|---|
| | Re0 | Re(40) | Re(−40) | Surface conditions | test |
| | nm | Nm | nm | — | — |
| Example 1 | 59.2 | 103.4 | 105.2 | ◯ | ◯ |
| Example 2 | 59.4 | 104.5 | 106.1 | ◯ | ◯ |
| Comparative | 58.8 | 105.0 | 106.1 | ◯ | ◯ |
| Example 1 | | | | | |

As seen in Table 2, Examples 1 and 2 showed larger values of Re0 of the optically anisotropic layer as compared with Comparative Example 1, proving desirable optical characteristics. It is supposedly because the irradiation of polarized ultraviolet light under the conditions of Examples 1 and 2 within the scope of the present invention could localize the radicals generated by the irradiation from the dichroic polymerization initiator, so that the polymerization could proceed in a localized manner, a sufficient level of distortion of the cholesteric alignment was produced, and thereby desirable optical characteristics were obtained.

### Comparison of Examples 1 to 5

### Energy Density

Next, the samples of Examples 3 to 5 were subjected to evaluation of surface conditions and rubbing test, similarly to as described in the above. Results are shown Table 3, together with the results of Examples 1 and 2.

TABLE 3

| | Surface | |
|---|---|---|
| | conditions | Rubbing test |
| | — | — |
| Example 1 | ◯ | ◯ |
| Example 2 | ◯ | ◯ |
| Example 3 | ◯ | Δ |
| Example 4 | ◯ | Δ |
| Example 5 | Δ | X |

In Examples 3 to 5, the percentage of components of light having extinction ratios ranging from 1 to 8 was 5%, which is in a desirable range for irradiation of polarized ultraviolet light, but the energy density became an insufficient level due to narrowed slit width, and consequently resulted in strength of the optically anisotropic layer lower than that in Examples 1 and 2. In particular, it is understandable that Example 5, using a dielectric mirror polarizer having a poor availability of light, was still lower in the energy density, and lower in the strength.

Although not shown in Table 3, optical characteristics (Re0, Re(40) and Re(−40)) of the optically anisotropic layer formed in Examples 3 to 5 were inferior to those in Example 1 and 2. It was, however, found possible to form the optically anisotropic layer having optical characteristics equivalent to those of Examples 1 and 2, by slowing the feeding speed.

### Comparison of Examples 6 to 9

### Surface Temperature of Film

Surface temperature of the coated film formed by coating the coating liquid for forming the optically anisotropic layer was kept under the conditions listed in Table 4, and irradiated with polarized UV shown for Example 2 in Table 1 (Examples 6 to 9). Conditions other than the surface temperature of film were same as those for Example 2. The obtained optically anisotropic layers were subjected to evaluation of surface conditions. Results are shown in Table 4.

TABLE 4

| | Surface | |
|---|---|---|
| | temperature of | Surface |
| | film | conditions |
| | ° C. | — |
| Example 6 | 90 | ◯ |
| Example 2 | 80 | ◯ |
| Example 7 | 60 | Δ |
| Example 8 | 25 | X |
| Example 9 | 50 | X |

It is understandable from data shown in Table 4, that lower surface temperatures in the process of polarized UV irradiation tends to induce disturbance in the alignment, and that the temperature is preferably as close to the isotropic phase transition temperature of the liquid crystal compound employed therein, in terms of obtaining desirable surface conditions.

Although not shown in Table 4, measurement of the optical characteristics (Re0, Re(40) and Re(−40)) of the optically anisotropic layers formed in Examples 6 to 9 showed that optically anisotropic layer formed in Example 6 was excellent in the optical characteristics similarly to those of optically anisotropic layer formed in Example 2, but the optical characteristics of the optically anisotropic layers formed in Examples 8 and 9 were poorer as compared with Example 2. It is therefore understandable that irradiation of polarized ultraviolet light is preferably carried out, while keeping the surface temperature of film close to the isotropic phase transition temperature, in terms of obtaining desirable optical characteristics.

### Comparison of Examples 10 to 15

### Irradiation for Post-Treatment

Ultraviolet irradiation was carried out according to the conditions listed in Table 5, and according to combinations listed in Table 6.

The optically anisotropic layers were formed under the same conditions with Example 1, except that conditions of the polarized ultraviolet irradiation were modified, such as replacing the conditions for ultraviolet irradiation with the conditions A or B below, such as carrying out the irradiation a plurality of number of times, and such as carrying out irradiation for post-treatment according to the irradiation condition C below.

TABLE 5

| | | Ratio of | | |
|---|---|---|---|---|
| | | components of | | |
| | | light with extinction | Energy | Feeding |
| | | ratio of 1 to 8 | density | speed |
| | Polarization | % | mJ/cm² | m/min |
| Irradiation | Yes | 7 | 150 | 10 |
| condition A | | | | |
| Irradiation | Yes | 7 | 75 | 20 |
| condition B | | | | |
| Irradiation | No | — | 350 | 5 |
| condition C | | | | |

TABLE 6

| | 1st irradiation | 2nd irradiation |
|---|---|---|
| Example 10 | Example 1 | Irradiation condition C |
| Example 11 | Example 2 | Irradiation condition C |
| Example 12 | Irradiation condition A | Irradiation condition C |
| Example 13 | Irradiation condition B | Irradiation condition C |
| Example 14 | Irradiation condition A | — |
| Example 15 | Irradiation condition B | — |

The samples were subjected to evaluation of optical characteristics, evaluation of surface conditions, and rubbing test. Results are shown in Table 7 below.

TABLE 7

| | Re0 | Re(40) | Re(−40) | | Rubbing |
|---|---|---|---|---|---|
| | (nm) | (nm) | (nm) | Surface conditions | test |
| Example 10 | 59.4 | 102.3 | 105.7 | ◯ | ⊚ |
| Example 11 | 61.8 | 109.9 | 107.8 | ◯ | ⊚ |
| Example 12 | 52.2 | 96.4 | 98.0 | ◯ | ◯ |
| Example 13 | 45.3 | 83.4 | 82.2 | ◯ | ◯ |
| Example 14 | 37.4 | 78.0 | 78.8 | Δ | Δ |
| Example 15 | 34.0 | 72.3 | 72.9 | Δ | X |

It is understandable from the data shown in Table 7, that the alignment and hardness were improved by the non-polarized UV irradiation after the polarized UV irradiation. Surprisingly, Examples showed improving tendencies also in the retardation.

### Example 16

### Manufacture of Polarizer Plate with Optical Compensation Film

The optical film produced in Example 1, and commercial Fujitac TD80UF (from Fujifilm Corporation, Re=3 nm, Rth=50 nm) were immersed in a 1.5 mol/L aqueous sodium hydroxide solution at 55° C. for 2 minutes. The films were then washed in a water bath at room temperature, and then neutralized at 30° C. using a 0.05 mol/L sulfuric acid. The films were washed again in a water bath at room temperature, and further dried under hot air of 100° C. The process was followed by washing with water and neutralization, and two thus-obtained saponified films were bonded roll-to-roll on both surfaces of the polarizer film, as the protective films for the polarizer plate, using a polyvinyl alcohol-base adhesive, to thereby manufacture an integrated polarizer plate.

### Example 17

### Manufacture and Evaluation of VA-Mode Liquid Crystal Display Device

The upper and lower polarizer plates of a commercial VA-LCD (SyncMaster 173P, from Samsung Electronics Co., Ltd.) were peeled off, a general polarizer plate was bonded to the upper side, and the polarizer plate produced in Example 16, having the optical film of Example 1, was bonded to the lower side so that the optically anisotropic layer is faced to the glass surface of the liquid crystal cell substrate, using a pressure-sensitive adhesive, to thereby manufacture the liquid crystal display device of the present invention. A schematic sectional view of thus-produced liquid crystal display device is shown in FIG. 11, together with angular relations of the individual optical axes. In FIG. 11, reference numeral 41 stands for a polarizer layer, 42 for a transparent support, 43 for an alignment layer, 44 for an optically anisotropic layer (42 to 44 express the optical film produced in Example 1), 45 for a polarizer plate protective film, 46 for a glass substrate for liquid crystal cell, 47 for a liquid crystal cell, and 48 for a pressure-sensitive adhesive layer. The arrow in the polarizer layer 41 indicates the direction of absorption axis, the arrows in the optically anisotropic layer 44, the support 44 thereof and the protective film 45 indicate the direction of slow axes, and the circle indicates that the arrow aligns in the direction of normal line of the sheet of drawing.

(Evaluation of VA-Mode Liquid Crystal Display Device)

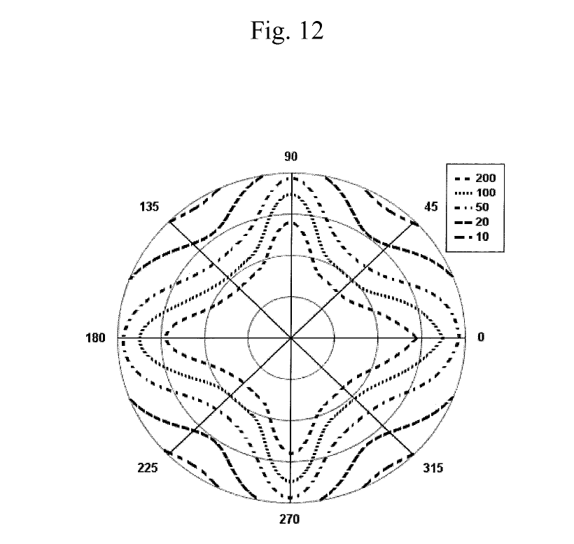

The viewing angle dependence of thus-produced liquid crystal display device was measured using a viewing angle meter (EZ Contrast 160D, from ELDIM). The device was also visually evaluated in particular in the direction of 45° C. inclination. Contrast characteristics of Example 2 measured by EZ Contrast were shown in FIG. 12, and result of visual observation was shown below.

| Sample | Result of Visual Evaluation |
|---|---|
| Example 17 | Only a small misalignment of color both inthe white state |
| | and in the black state, with desirable gradation |
| | characteristics of middle tone. |

### Example 18

### Manufacture of Transfer Material

The optical film was produced similarly to as in Example 1. Exceptions were such as using, in place of the transparent support S-1 used in Example 1, a rolled temporary support composed of a polyethylene terephthalate film of 75 μm thick, having thereon thermoplastic polymer layer (of 14.6 μm thick) formed by coating and drying the coating liquid CU-1 for forming the thermoplastic polymer layer using a slit-form nozzle, and such that the alignment layer (of 1.6 μm thick) was formed by coating and drying the coating liquid AL-2 for forming the intermediate layer/alignment layer. Except for the above, the optical film was produced by forming the optically anisotropic layer under the conditions completely similar to those in Example 1. Next, the photosensitive polymer composition PP-1 was coated and dried on the surface of thus-formed optically anisotropic layer, to thereby form the photosensitive polymer layer, and thereby the transfer material of the present invention was produced.

Using a laminator (Lamic II from Hitachi Plant Technologies, Ltd.), the photosensitive polymer transfer material was stacked on the surface of the substrate preheated at 100° C. for 2 minutes, so that the photosensitive polymer layer is faced to the surface of the substrate, and laminated at a rubber roller temperature of 130° C., a line pressure of 100 N/cm, and a feeding speed of 2.2 m/min, the temporary support was separated, and the product was exposed over the entire surface thereof using a ultrahigh pressure mercury lamp at an energy of exposure of 50 mJ/cm². The product was further baked at 240° C. for 2 hours, to thereby manufacture a glass substrate for VA-LCD.

Next, using “Transer” system (from Fujifilm Corporation) described in FUJIFILM RESEARCH & DEVELOPMENT No. 44, p. 25 (1999), a black matrix and R, G, B color filters were formed on the glass substrate.

(Formation of Transparent Electrodes)

On the color filters formed as described in the above, a transparent electrode layer was formed by sputtering of ITO.

(Manufacture of Photosensitive Transfer material for Forming Projections)

On a temporary support composed of a polyethylene terephthalate film of 75 μm thick, the coating liquid TP-1 for forming the thermoplastic polymer layer was coated and dried, to thereby form a thermoplastic polymer layer having a dry thickness of 15 μm.

Next, on the thermoplastic polymer layer, the coating liquid AL-2 for forming the intermediate layer/alignment layer was coated and dried, to thereby form an intermediate layer having a dry thickness of 1.6 μm.

On the intermediate layer, a coating liquid having the composition below was coated and dried, to thereby form a photosensitive polymer layer for forming the projections for controlling alignment of liquid crystal, having a dry thickness of 2.0 μm.

| Formulation of Coating Liquid for Forming Projections | (%) |
|---|---|
| FH-2413F (from Fujifilm Arch Co., Ltd.) | 53.3 |
| Methyl ethyl ketone | 46.66 |
| Megafac F-176PF | 0.04 |

Further on the surface of the photosensitive polymer layer, a polypropylene film of 12 μm thick was bonded as a cover film, to thereby manufacture a transfer material having, on the temporary support, the thermoplastic polymer layer, the intermediate layer, the photosensitive polymer layer and the cover film stacked in this order.

(Formation of Projections)

The transfer material for forming projections produced as described in the above was removed with the cover film, and stacked on the surface of the color-filter-side substrate, so that the surface of the photosensitive polymer layer thereof is faced to the side of the substrate having the ITO film formed thereon, and the stack was laminated using a laminator (Lamic II, from Hitachi Plant Technologies, Ltd.) at a line pressure of 100 N/cm, a temperature of 130° C., and a feeding speed of 2.2 m/min. Only the temporary support was then separated from the transfer material at the boundary with the thermoplastic polymer layer, and removed. In this state, the product has, on the color-filter-side substrate, the photosensitive polymer layer, the intermediate layer, and the thermoplastic polymer layer stacked in this order.

Next, a proximity exposure apparatus was disposed over the topmost thermoplastic polymer layer, so as to locate the photomask thereof 100 μm up away from the surface of the photosensitive polymer layer, and the stack was subjected to proximity exposure through the photomask using a ultrahigh pressure mercury lamp at an energy of exposure of 70 mJ/cm². The substrate was sprayed with an 1% aqueous triethanolamine solution using a shower-type developing apparatus at 30° C. for 30 seconds, to thereby remove, by dissolution, the thermoplastic polymer layer and the intermediate layer. Up to this stage, the photosensitive polymer layer was found to remain substantially undeveloped.

Succeedingly, the development was continued by spraying an aqueous solution containing 0.085 mol/L of sodium carbonate, 0.085 mol/L or sodium hydrogen carbonate, and 1% of sodium dibutylnaphthalene sulfonate, using the shower-type developing apparatus at 33° C. for 30 seconds, to thereby remove, by dissolution, the unnecessary portion (uncured portion) of the photosensitive polymer layer. By this process, projections composed of photosensitive polymer layer were formed on the color-filter-side substrate, as being patterned with a predetermined geometry. Next, the color-filter-side substrate having the projections formed thereon was baked at 240° C. for 50 minutes, to thereby form the projections for controlling alignment of liquid crystal, having a height of 1.5 μm, and a semicylindrical section on the color-filter-side substrate.

(Formation of Alignment Layer)

Further thereon an alignment film composed of polyimide was formed. A sealing material composed of an epoxy polymer containing spacer particles was then printed on the color-filter-side substrate at the position corresponded to the outer frame of the black matrix provided around the pixel groups having the color filters, and the color-filter-side substrate and the opposed substrate were bonded under a pressure of 10 kg/cm. Next, thus-bonded glass substrates were annealed at 150° C. for 90 minutes so as to cure the sealing material, to thereby obtain the stack of two glass substrates. The stack of the glass substrates were degassed in vacuo, and a liquid crystal was injected into the gap between two glass substrates by recovering the atmospheric pressure, to thereby obtain the liquid crystal cell. On both surfaces of the liquid crystal cell, polarizer plates HLC2-2518 from Sanritz Corporation were bonded.

(Manufacture of VA-LCD)

As a backlight for a cold cathode ray tube of a color liquid crystal display device, a white three-band phosphor type fluorescent lamp having an arbitrary hue was produced using a 50:50, by mass, mixture of BaMg₂Al₁₆O₂₇:Eu,Mn and LaPO₄:Ce,Tb as a green phosphor (G), Y₂O₃:Eu as a red phosphor (R), and BaMgAl₁₀O₁₇:Eu as a blue phosphor (B). On the backlight, the liquid crystal cell bonded with the polarizer plates as described in the above was disposed, to thereby manufacture a VA-LCD.

(Evaluation of VA-LCD)

Leakage of light in the black state (under no applied voltage) of thus-produced liquid crystal display device, in particular at the corner portions thereof, was evaluated by visual observation firstly at room temperature, and then observed again after allowing the LCD to stand in a thermostat-hygrostat condition of 40° C., 90% RH for 48 hours. Result is shown below.

| Sample | Result of Visual Evaluation |
|---|---|
| Example 18 | The black state remained almost unchanged, |
| | showing no distinctive leakage of light at the corners. |

### INDUSTRIAL APPLICABILITY

According to the present invention, it is possible to provide a method of producing an optical film, including a step of irradiating polarized ultraviolet light, capable of producing an optical film having desirable optical characteristics and strength of the film, with an excellent productivity.

According to the present invention, it is also possible to provide a method of producing an optical film contributive to improvement in the viewing angle dependence of liquid crystal display devices, in particular VA-mode liquid crystal display devices, in a continuous and stable manner, with no, or minimum failures.

According to the present invention, it is also possible to provide a polarizer plate having such optical film and applicable as one component of liquid crystal display devices, in particular VA-mode liquid crystal display devices, and a transfer material allowing simple formation of an optically anisotropic layer in liquid crystal cells.

According to the present invention, it is also possible to provide a liquid crystal display device, in particular VA-mode liquid crystal display device having the liquid crystal cell thereof optically compensated in an exact manner, possibly thinned, and excellent in the viewing angle dependence.

What is claimed is:

1. A method of producing an optical film comprising steps (1) to (3) in this order:
(1) preparing, on a surface of an alignment film, a layer of a polymerizable composition comprising a polymerizable liquid crystal compound and a dichroic polymerization initiator;
(2) aligning molecules of said polymerizable liquid crystal compound in said layer in a first alignment state; and
(3) irradiating said layer with polarized ultraviolet light to carry out polymerization of said polymerizable liquid crystal compound and fix molecules of said polymerizable liquid crystal compound in a second alignment state thereby to form an optically anisotropic layer,
wherein a percentage of polarized ultraviolet light having an extinction ratio ranging from 1 to 8 is not greater than 15% with respect to an energy density of polarized ultraviolet light per unit area (J/cm²).

2. The method of claim 1, wherein a surface temperature of said layer in the step (3) is from (Tᵢₛₒ−50) to Tᵢₛₒ° C. (where, Tᵢₛₒ(° C.) is isotropic phase transition temperature of said polymerizable liquid crystal compound).

3. The method of claim 1, wherein, in the step (3), polarized ultraviolet light is irradiated with an energy density within the range from 200 mJ/cm² to 2 J/cm².

4. The method of claim 1, wherein the layer is irradiated with non-polarized ultraviolet light after the step (3).

5. A polarized ultraviolet exposure apparatus to be used in a method as set forth in claim 1, comprising:
an ultraviolet radiation source;
a unit of converting non-polarized ultraviolet light from said radiation source into polarized ultraviolet light; and
a unit of preventing an object to be irradiated from being irradiated with polarized ultraviolet light having an extinction ratio ranging from 1 to 8.

6. An optical film produced by a method as set forth in claim 1.

7. A polarizer plate comprising a polarizer film, and an optical film as set forth in claim 6.

8. A transfer material comprising:
an optical film produced according to a method as set forth in claim 1; and
a photosensitive polymer layer disposed on an optically anisotropic layer of said optical film.

9. A liquid crystal display device comprising at least one selected from a polarizer plate as set forth in claim 7, an optical film as set forth in claim 6, and an optically anisotropic layer transferred from a transfer material as set forth in claim 8.

10. A liquid crystal display device comprising, in a liquid crystal cell thereof, an optically anisotropic layer transferred from a transfer material as set forth in claim 8.

11. The liquid crystal display device of claim 9, employing a VA-mode as a display mode.

12. The liquid crystal display device of claim 10, employing a VA-mode as a display mode.
